(19) United States
(12) Patent Application Publication
(10) Pub. No.: US 2023/0233519 A1
(43) Pub. Date: Jul. 27, 2023

(54) COMBINATION THERAPIES

(71) Applicant: MEI Pharma, Inc., San Diego, CA (US)

(72) Inventor: Daniel P. GOLD, San Diego, CA (US)

(21) Appl. No.: 18/001,341

(22) PCT Filed: Jun. 10, 2021
(86) PCT No.: PCT/US2021/036833

§ 371 Date: Dec. 9, 2022

Related U.S. Application Data
(60) Provisional application No. 63/037,750, filed on Jun. 11, 2020.

(51) Int. Cl. A61K 31/353; A61K 31/635; A61K 45/06

(52) U.S. Cl. CPC A61K 31/353; A61K 31/635; A61K 45/06

(57) ABSTRACT

Provided herein are compositions, methods, and kits for treating cancer comprising a mitochondrial inhibitor and a Bcl-2 inhibitor.

### CROSS-REFERENCE

This application claims the benefit of U.S. Provisional Patent Application No. 63/037,750, filed Jun. 11, 2020, which is incorporated herein by reference in its entirety.

### BACKGROUND OF THE INVENTION

Cancer is the leading cause of death worldwide. Novel, efficacious therapies are provided herein to address an unmet need in the treatment of various cancers.

### SUMMARY OF THE INVENTION

Provided herein are pharmaceutical compositions, methods of treating disease, and kits. Provided in certain embodiments herein is a composition, wherein the composition comprises an oxidative phosphorylation inhibitor and a Bcl-2 family inhibitor. In some embodiments, the composition further comprises a pharmaceutically acceptable excipient. In some embodiments, the composition comprises a liquid vehicle(s) to provide a physiologically acceptable formulation for parenteral administration. Also provided herein are combination therapies, comprising administration of an oxidative phosphorylation inhibitor and a Bcl-2 family inhibitor to an individual in need thereof. In some embodiments, the oxidative phosphorylation inhibitor is a mitochondrial oxygenase inhibitor. In some embodiments, the oxidative phosphorylation inhibitor is a benzopyran derivative. In some embodiments, the combination therapy comprises administration of a benzopyran derivative and a Bcl-2 family inhibitor. Some embodiments of the present invention provide a method for the treatment of cancer comprising administration of the composition to an individual in need of cancer therapy.

Some embodiments provided herein describe a method of treating cancer, comprising administering to a subject in need thereof an effective amount of:

(i) a Bcl-2 family inhibitor; and

(ii) a compound of Formula (II) or a pharmaceutically acceptable salt thereof:

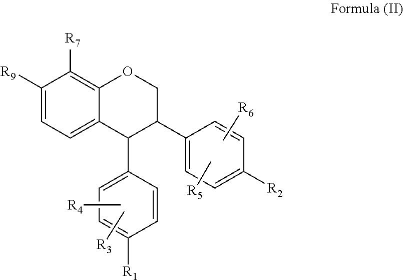

wherein
R₁ is hydroxy, alkoxy, haloalkyl, or halo;
R₂ is hydroxy or alkoxy;
R₃, R₄, R₅, and R₆ are independently hydrogen, hydroxy, alkoxy, halo, haloalkyl, or alkyl;
R₇ is alkyl or hydrogen; and
R₉ is hydroxy or alkoxy.

In some embodiments, R₁ is hydroxy or alkoxy. In some embodiments, R₂ is hydroxy. In some embodiments, R₃, R₄, R₅, and R₆ are independently hydrogen or alkyl. In some embodiments, R₄, R₅, and R₆ are independently hydrogen. In some embodiments, R₇ is methyl or hydrogen. In some embodiments, R₁ is hydroxy or alkoxy, R₂ is hydroxy or alkoxy, R₃, R₄, R₅, and R₆ are independently hydrogen, hydroxy, alkoxy, or alkyl, R₇ is alkyl or hydrogen, and R₉ is hydroxy or alkoxy. In other embodiments, R₁ is hydroxy or alkoxy, R₂ is hydroxy or alkoxy, R₃, R₄, R₅, and R₆ are independently hydrogen, R₇ is alkyl or hydrogen, and R₉ is hydroxy. In some embodiments, R₁ is hydroxy or methoxy, R₂ is hydroxy or methoxy, R₃, R₄, R₅, and R₆ are independently hydrogen, hydroxy, methoxy, methyl, R₇ is methyl or hydrogen, and R₉ is hydroxy or methoxy. In other embodiments, R₁ is hydroxy or methoxy, R₂ is hydroxy or methoxy, R₃, R₄, R₅, and R₆ are independently hydrogen, R₇ is methyl or hydrogen, and R₉ is hydroxy.

In some embodiments, the compound of Formula (II) is 3-(4-hydroxyphenyl)-4-(4-hydroxyphenyl)-8-methylchroman-7-ol. In other embodiments, the compound of Formula (II) is cis-3-(4-hydroxyphenyl)-4-(4-hydroxyphenyl)-8-methylchroman-7-ol. In still other embodiments, the compound of Formula (II) is d-cis-3-(4-hydroxyphenyl)-4-(4-hydroxyphenyl)-8-methylchroman-7-ol.

In some embodiments, the Bcl-2 family inhibitor is an inhibitor of Bcl-2, Bcl-xL, Bcl-x, Bcl-w, Bcl-b, BH3-only, or MCL-1. In some embodiments, the Bcl-2 family inhibitor is an inhibitor of Bcl-2. In specific embodiments, the Bcl-2 family inhibitor is a BH3-mimetic. In some embodiments, the Bcl-2 family inhibitor is venetoclax, navitoclax, obatoclax mesylate, ABT-737, APG 2575, APG 1252, or AT-101. In some embodiments, the Bcl-2 family inhibitor is venetoclax.

In some embodiments, the cancer is leukemia, lymphoma, lung cancer, or a hematological malignancy. In some embodiments, cancer is a leukemia or lymphoma. In some embodiments, the leukemia is acute myeloid leukemia (AML), chronic myeloid leukemia (CML), acute lymphocytic leukemia (ALL), or chronic lymphocytic leukemia (CLL). In some embodiments, the leukemia is acute myeloid leukemia (AML). In other embodiments, the cancer is lung cancer. In some embodiments, the lung cancer is non-small cell lung cancer (NSCLC), small cell lung cancer (SCLC), a lung carcinoid tumor, or adenoid cystic carcinoma. In some embodiments, the non-small cell lung cancer (NSCLC) is lung adenocarcinoma, squamous cell carcinoma, large cell (undifferentiated) carcinoma, adenosquamous carcinoma, or sarcomatoid carcinoma. In some embodiments, the cancer is non-responsive or resistant to the Bcl-2 inhibitor. In some embodiments, the Bcl-2 family inhibitor and compound of formula (II) are administered simultaneously. In some embodiments, the Bcl-2 family inhibitor and compound of formula (II) are administered sequentially.

Some embodiments provided herein describe a method of treating leukemia comprising administering to a subject in need thereof an effective amount of

(i) a Bcl-2 family inhibitor; and

(ii) a compound of Formula (II) or a pharmaceutically acceptable salt thereof:

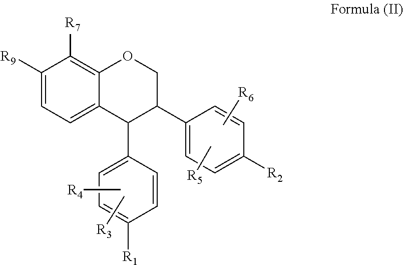

wherein
R₁ is hydroxy, alkoxy, haloalkyl, or halo;
R₂ is hydroxy or alkoxy;
R₃, R₄, R₅, and R₆ are independently hydrogen, hydroxy, alkoxy, halo, haloalkyl, or alkyl;
R₇ is alkyl or hydrogen; and
R₉ is hydroxy or alkoxy.

In some embodiments, the leukemia is acute myeloid leukemia (AML), chronic myeloid leukemia (CIVIL), acute lymphocytic leukemia (ALL), or chronic lymphocytic leukemia (CLL). In certain embodiments, the leukemia is acute myeloid leukemia (AML). In some embodiments, the Bcl-2 family inhibitor is venetoclax, navitoclax, obatoclax mesylate, ABT-737, APG 2575, APG 1252, or AT-101. In certain embodiments, the Bcl-2 family inhibitor is venetoclax.

In some embodiments, the compound of Formula (II) is 3-(4-hydroxyphenyl)-4-(4-hydroxyphenyl)-8-methylchroman-7-ol. In other embodiments, the compound of Formula (II) is cis-3-(4-hydroxyphenyl)-4-(4-hydroxyphenyl)-8-methylchroman-7-ol. In some embodiments, the compound of Formula (II) is d-cis-3-(4-hydroxyphenyl)-4-(4-hydroxyphenyl)-8-methylchroman-7-ol.

Some embodiments provided herein describe a method of treating lung cancer comprising administering to a subject in need thereof an effective amount of

(i) a Bcl-2 family inhibitor; and

(ii) a compound of Formula (II) or a pharmaceutically acceptable salt thereof:

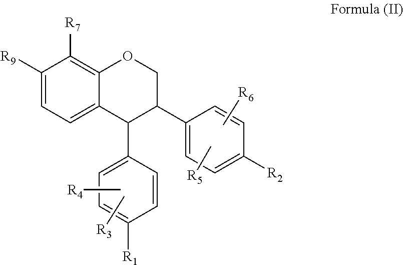

wherein
R₁ is hydroxy, alkoxy, haloalkyl, or halo;
R₂ is hydroxy or alkoxy;
R₃, R₄, R₅, and R₆ are independently hydrogen, hydroxy, alkoxy, halo, haloalkyl, or alkyl;
R₇ is alkyl or hydrogen; and
R₉ is hydroxy or alkoxy.

In some embodiments, the lung cancer is non-small cell lung cancer (NSCLC), small cell lung cancer (SCLC), a lung carcinoid tumor, or adenoid cystic carcinoma. In some embodiments, the non-small cell lung cancer (NSCLC) is lung adenocarcinoma, squamous cell carcinoma, large cell (undifferentiated) carcinoma, adenosquamous carcinoma, or sarcomatoid carcinoma. In some embodiments, the Bcl-2 family inhibitor is venetoclax, navitoclax, obatoclax mesylate, ABT-737, APG 2575, APG 1252, or AT-101. In some embodiments, the Bcl-2 family inhibitor is venetoclax. In some embodiments, the compound of Formula (II) is 3-(4-hydroxyphenyl)-4-(4-hydroxyphenyl)-8-methylchroman-7-ol. In other embodiments, the compound of Formula (II) is cis-3-(4-hydroxyphenyl)-4-(4-hydroxyphenyl)-8-methylchroman-7-ol. In still other embodiments, the compound of Formula (II) is d-cis-3-(4-hydroxyphenyl)-4-(4-hydroxyphenyl)-8-methylchroman-7-ol.

Some embodiments provided herein describe a pharmaceutical composition comprising:

(i) a Bcl-2 family inhibitor; and

(ii) a compound of Formula (II) or a pharmaceutically acceptable salt thereof:

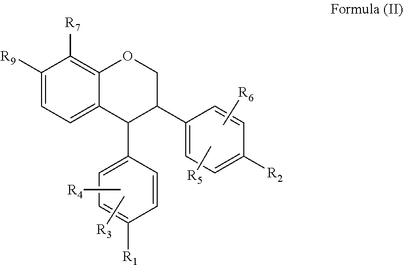

wherein
R₁ is hydroxy, alkoxy, haloalkyl, or halo;
R₂ is hydroxy or alkoxy;
R₃, R₄, R₅, and R₆ are independently hydrogen, hydroxy, alkoxy, halo, haloalkyl, or alkyl;
R₇ is alkyl or hydrogen; and
R₉ is hydroxy or alkoxy; and

(iii) a pharmaceutically acceptable excipient.

In some embodiments, the Bcl-2 family inhibitor is venetoclax, navitoclax, obatoclax mesylate, ABT-737, APG 2575, APG 1252, or AT-101. In some embodiments, the Bcl-2 family inhibitor is venetoclax. In some embodiments, the compound of Formula (II) is 3-(4-hydroxyphenyl)-4-(4-hydroxyphenyl)-8-methylchroman-7-ol. In other embodiments, the compound of Formula (II) is cis-3-(4-hydroxyphenyl)-4-(4-hydroxyphenyl)-8-methylchroman-7-ol. In still other embodiments, the compound of Formula (II) is d-cis-3-(4-hydroxyphenyl)-4-(4-hydroxyphenyl)-8-methylchroman-7-ol.

Some embodiments provided herein describe a kit comprising one or more containers filled with a Bcl-2 family inhibitor and one or more containers filled with a compound of Formula (II) or a pharmaceutically acceptable salt thereof:

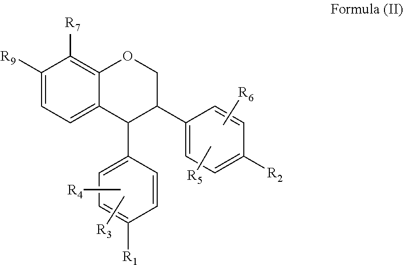

wherein
R₁ is hydroxy, alkoxy, haloalkyl, or halo;
R₂ is hydroxy or alkoxy;
R₃, R₄, R₅, and R₆ are independently hydrogen, hydroxy, alkoxy, halo, haloalkyl, or alkyl;
R₇ is alkyl or hydrogen; and
R₉ is hydroxy or alkoxy.

In some embodiments, the Bcl-2 inhibitor is venetoclax. In some embodiments, the at least one compound of Formula (II) is 3-(4-hydroxyphenyl)-4-(4-hydroxyphenyl)-8-methylchroman-7-ol. In some embodiments, the at least one compound of Formula (II) is cis-3-(4-hydroxyphenyl)-4-(4-hydroxyphenyl)-8-methylchroman-7-ol. In some embodiments, the at least one compound of Formula (II) is d-cis-3-(4-hydroxyphenyl)-4-(4-hydroxyphenyl)-8-methylchroman-7-ol.

### BRIEF DESCRIPTION OF THE FIGURES

FIG. 1A depicts apoptosis of MOLM-13 AML cells when MOLM-13 AML cell lines were treated with venetoclax (VEN) and d-cis-3-(4-hydroxyphenyl)-4-(4-hydroxyphenyl)-8-methylchroman-7-ol (Compound A), alone or in combination, for 24 h and then subjected to Annexin V-FITC/Pl staining and flow cytometry analysis. ***p<0.001. AnnexinV⁺/Pl⁻ represents early apoptotic cells and Annexin V⁺/Pl⁺ represent middle and late apoptotic cells or necrotic cells.

FIG. 1B depicts apoptosis of MV4-11 childhood AML cells when MV4-11 childhood AML cell lines were treated with venetoclax (VEN) and d-cis-3-(4-hydroxyphenyl)-4-(4-hydroxyphenyl)-8-methylchroman-7-ol (Compound A), alone or in combination, for 24 h and then subjected to Annexin V-FITC/Pl staining and flow cytometry analysis. ***p<0.001. AnnexinV⁺/Pl⁻ represents early apoptotic cells and Annexin V⁺/Pl⁺ represent middle and late apoptotic cells or necrotic cells.

FIG. 1C depicts apoptosis of THP-1 childhood AML cells when THP-1 childhood AML cell lines were treated with venetoclax (VEN) and d-cis-3-(4-hydroxyphenyl)-4-(4-hydroxyphenyl)-8-methylchroman-7-ol (Compound A), alone or in combination, for 24 h and then subjected to Annexin V-FITC/Pl staining and flow cytometry analysis. ***p<0.001. AnnexinV⁺/Pl⁻ represents early apoptotic cells and Annexin V⁺/Pl⁺ represent middle and late apoptotic cells or necrotic cells.

FIG. 1D depicts apoptosis of cytarabine resistant (araC-R) U937 acute monocytic leukemia (AMoL) cells when araC-R U937 cells were treated with venetoclax (VEN) and d-cis-3-(4-hydroxyphenyl)-4-(4-hydroxyphenyl)-8-methylchroman-7-ol (Compound A), alone or in combination, for 24 h and then subjected to Annexin V-FITC/Pl staining and flow cytometry analysis. ***p<0.001. AnnexinV⁺/Pl⁻ represents early apoptotic cells and Annexin V⁺/Pl⁺ represent middle and late apoptotic cells or necrotic cells.

FIG. 2A depicts the percentage of viable MOLM-13 AML cells following treatment of MOLM-13 AML cell lines with venetoclax (VEN) and d-cis-3-(4-hydroxyphenyl)-4-(4-hydroxyphenyl)-8-methylchroman-7-ol (Compound A), alone or in combination, for 24 h and then subjected to Annexin V-FITC/PI staining and flow cytometry analysis. Viable cells (annexin V negative and PI negative) were gated. Viable cell counts from no drug treatment controls were set at 100%, and the rest of the samples were normalized to the no drug treatment controls. The data were presented as mean percent of viable cells from triplicates with standard errors.

FIG. 2B depicts the percentage of viable MV4-11 childhood AML cells following treatment of MV4-11 childhood AML cell lines with venetoclax (VEN) and d-cis-3-(4-hydroxyphenyl)-4-(4-hydroxyphenyl)-8-methylchroman-7-ol (Compound A), alone or in combination, for 24 h. and then subjected to Annexin V-FITC/PI staining and flow cytometry analysis. Viable cells (annexin V negative and PI negative) were gated. Viable cell counts from no drug treatment controls were set at 100%, and the rest of the samples were normalized to the no drug treatment controls. The data were presented as mean percent of viable cells from triplicates with standard errors.

FIG. 2C depicts the percentage of viable THP-1 childhood AML cells following treatment of THP-1 childhood AML cell lines with venetoclax (VEN) and d-cis-3-(4-hydroxyphenyl)-4-(4-hydroxyphenyl)-8-methylchroman-7-ol (Compound A), alone or in combination, for 24 h. and then subjected to Annexin V-FITC/PI staining and flow cytometry analysis. Viable cells (annexin V negative and PI negative) were gated. Viable cell counts from no drug treatment controls were set at 100%, and the rest of the samples were normalized to the no drug treatment controls. The data were presented as mean percent of viable cells from triplicates with standard errors.

FIG. 3 depicts the percentage of viable cells in drug-resistant H596 lung cancer cell lines following treatment with d-cis-3-(4-hydroxyphenyl)-4-(4-hydroxyphenyl)-8-methylchroman-7-ol (Compound A), alone or in combination with venetoclax (VEN) for 48 hours. Cell viability was measured by MTT assay. Viability (% live cells) was calculated relative to untreated control cells.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

There is a continuing need to develop and provide effective therapies for the treatment of cancer. Described herein are combination compositions and combination therapies for the treatment of cancer. The compositions and therapies described herein comprise a benzopyran derivative (e.g., substituted diaryl chroman derivatives) and Bcl-2 family inhibitors. Also provided herein are methods of treating leukemia, methods of treating lung cancer, and kits comprising one or more containers filled with a benzopyran derivative (e.g., of Formula II) and a Bcl-2 family inhibitor.

### Certain Definitions

Unless otherwise noted, terminology used herein should be given its normal meaning as understood by one of skill in the art.

The term “alkyl” as used herein, alone or in combination, refers to an optionally substituted straight-chain, or optionally substituted branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁-6 alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated.

The terms “C₁-C₃-alkyl” and “C₁-C₆-alkyl” as used herein refer to saturated, straight- or branched-chain hydrocarbon radicals derived from a hydrocarbon moiety containing between one and three, one and six, and one and twelve carbon atoms, respectively, by removal of a single hydrogen atom. Examples of C₁-C₃-alkyl radicals include methyl, ethyl, propyl and isopropyl. Examples of C₁-C₆-alkyl radicals include, but are not limited to, methyl, ethyl, propyl, isopropyl, n-butyl, tert-butyl, neopentyl and n-hexyl.

The term “cycloalkyl” as used herein refers to a monovalent group derived from a monocyclic or bicyclic saturated carbocyclic ring compound containing between three and twenty carbon atoms by removal of a single hydrogen atom.

The term “C₃-C₆ cycloalkyl” denotes a monovalent group derived from a monocyclic or bicyclic saturated carbocyclic ring compound by removal of a single hydrogen atom. Examples include cyclopropyl, cyclobutyl, cyclopentyl, and cyclohexyl.

The alkyl group or cycloalkyl group may optionally be substituted by one or more of fluorine, chlorine, bromine, iodine, carboxyl, C₁₋₄ alkoxycarbonyl, C₁₋₄ alkylaminocarbonyl, di-(C₁₋₄ alkyl)-aminocarbonyl, hydroxyl, C₁₋₄ alkoxy, formyloxy, C₁₋₄ alkylcarbonyloxy, C₁₋₄ alkylthio, C₃₋₆ cycloalkyl or phenyl.

The term “alkoxy” as used herein, alone or in combination, refers to an alkyl ether radical, —O-alkyl, including the groups —O-aliphatic and —O-carbocyclyl, wherein the alkyl, aliphatic and carbocyclyl groups may be optionally substituted, and wherein the terms alkyl, aliphatic and carbocyclyl are as defined herein. Non-limiting examples of alkoxy radicals include methoxy, ethoxy, n-propoxy, isopropoxy, n-butoxy, iso-butoxy, sec-butoxy, tert-butoxy and the like.

The terms “C₁-C₃-alkoxy” and “C₁-C₆-alkoxy” as used herein refers to the C₁-C₃-alkyl group and C₁-C₆-alkyl group, as previously defined, attached to the parent molecular moiety through an oxygen atom. Examples of C₁-C₆-alkoxy radicals include, but are not limited to, methoxy, ethoxy, propoxy, isopropoxy, n-butoxy, tert-butoxy, neopentoxy and n-hexoxy.

The terms “halo” and “halogen” as used herein refer to an atom selected from fluorine, chlorine, bromine and iodine.

The term “haloalkyl” includes “alkyl” wherein one or more such as 1, 2, 3, 4, or 5 of the hydrogens have been replaced by a halo atom. The haloalkyl may be straight chain or branched chain “alkyl” unit. Non-limiting examples include —CH₂F, —CHF₂, —CF₃, —CH₂CH₂F, —CH₂CHF₂, —CH₂CF₃, —CF₂CH₂F, —CF₂CHF₂, —CF₂CF₃, —CH₂Cl, —CHCl₂, —CCl₃, —CH₂Br, —CHBr₂, and —CBr₃.

The term “fluoroalkyl” includes “alkyl” wherein one or more such as 1, 2, 3, 4, or 5 of the hydrogens have been replaced by fluoro. The fluoroalkyl may be straight chain or branched chain “alkyl” unit. Preferred fluoroalkyl groups include trifluoromethyl and pentafluoroethyl.

The term “acceptable” with respect to a formulation, composition, or ingredient, as used herein, means having no persistent detrimental effect on the general health of the subject being treated.

The term “pharmaceutically acceptable”, as used herein, refers to a material, including but is not limited, to a salt, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. For example, S. M. Berge, et al. describes pharmaceutically acceptable salts in detail in J. Pharmaceutical Sciences, 66: 1-19 (1977), incorporated herein by reference for this purpose. The salts are prepared in situ during the final isolation and purification of the compounds described herein, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

It should be understood that a reference to a salt includes the solvent addition forms or crystal forms thereof, particularly solvates or polymorphs. Solvates contain either stoichiometric or non-stoichiometric amounts of a solvent, and are often formed during the process of crystallization with pharmaceutically acceptable solvents such as water, ethanol, and the like. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The term “cyclodextrin,” as used herein, refers to cyclic carbohydrates consisting of at least six to eight sugar molecules in a ring formation. The outer part of the ring contains water soluble groups; at the center of the ring is a relatively nonpolar cavity able to accommodate small molecules.

The terms “administer,” “administering,” “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. An appropriate “effective” amount in any individual case may be determined using techniques, such as a dose escalation study.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “patient”, “subject” or “individual” are used interchangeably. As used herein, they refer to individuals suffering from a disorder, and the like, encompasses mammals and non-mammals. None of the terms require that the individual be under the care and/or supervision of a medical professional. Mammals are any member of the Mammalian class, including but not limited to humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the individual is a mammal. In preferred embodiments, the individual is a human.

The terms “treat”, “treating” or “treatment”, as used herein, include alleviating, abating or ameliorating a disease or condition or one or more symptoms thereof, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The terms “preventing” or “prevention” refer to a reduction in risk of acquiring a disease or disorder (i.e., causing at least one of the clinical symptoms of the disease not to develop in a subject that may be exposed to or predisposed to the disease but does not yet experience or display symptoms of the disease).

The term “carrier” as used herein, refers to relatively nontoxic chemical compounds or agents that facilitate the incorporation of a compound into cells or tissues.

A dosage of an oxidative phosphorylation inhibitor or a Bcl-2 family inhibitor may be expressed in absolute or relative terms. For example, a dosage of either an oxidative phosphorylation inhibitor or a Bcl-2 family inhibitor may be expressed as a certain number of milligrams (mg) of an oxidative phosphorylation inhibitor or a Bcl-2 family inhibitor, or a pharmaceutically acceptable salt thereof, administered to a patient. In relative terms, a dosage of an oxidative phosphorylation inhibitor or a Bcl-2 family inhibitor herein may be expressed as “mg/kg,” which expresses the number of milligrams of the oxidative phosphorylation inhibitor or Bcl-2 family inhibitor, or pharmaceutically acceptable salt thereof, administered to a patient per kg of the patient's body weight. Dosage may also be expressed in terms of mg/m², indicating the mass of active ingredient administered per square meter of the patient's estimated surface area.

### Benzopyran Derivative Compounds

Some embodiments of the present invention describe benzopyran derivatives. In some embodiments, the benzopyran derivative is a substituted diaryl chroman derivative, super-benzopyrans, or a combination thereof. In some embodiments, the benzopyran derivative is an oxidative phosphorylation inhibitor. In some embodiments, the oxidative phosphorylation inhibitor is an inhibitor of a mitochondrial complex involved in the electron transport chain, e.g., complex I, II, III, or IV. In some embodiments, the oxidative phosphorylation inhibitor is an inhibitor of mitochondrial complex I. In some embodiments, the mitochondrial complex I inhibitor inhibits the production of ATP in the mitochondria. In some embodiments, the mitochondrial complex I inhibitor induces cell death (e.g., in a cancer cell). In some embodiments, the mitochondrial complex I inhibitor selectively induces cell death in a cancer cell. In some embodiments, the mitochondrial complex I inhibitor induces cell death in a cancer cell (e.g., via destructive autophagy) that is sensitive or sensitized to disruptions in mitochondrial metabolism. In some embodiments, the mitochondrial complex I inhibitor induces cell death in an oxidative phosphorylation reliant cancer cell. In some embodiments, the mitochondrial complex I inhibitor is a benzopyran derivative described herein.

Some embodiments of the present invention describe a benzopyran derivative having the structure of Formula (II):

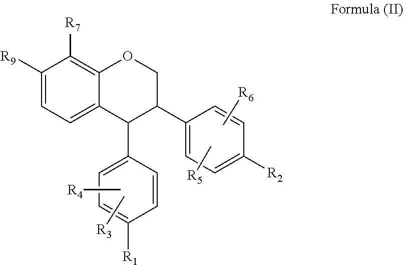

R₁ is hydroxy, alkoxy, haloalkyl, or halo;
R₂ is hydroxy or alkoxy;
R₃, R₄, R₅, and R₆ are independently hydrogen, hydroxy, alkoxy, halo, haloalkyl, or alkyl and
R₇ is alkyl or hydrogen; and
R₉ is hydroxy or alkoxy;
or a pharmaceutically acceptable salt thereof.

In some embodiments, R₁ is hydroxy or alkoxy. In some embodiments, R₁ is hydroxy. In other embodiments, R₁ is C₁-C₆alkoxy. In further or additional embodiments, R₁ is C₁-C₃alkoxy. In other embodiments, R₁ is C₁-C₂alkoxy. In specific embodiments, R₁ is methoxy. In specific embodiments, R₁ is ethoxy. In specific embodiments, R₁ is propoxy. In specific embodiments, R₁ is iso-propoxy. In specific embodiments, R₁ is butoxy. In specific embodiments, R₁ is iso-butoxy. In specific embodiments, R₁ is sec-butoxy. In specific embodiments, R₁ is tert-butoxy. In specific embodiments, R₁ is pentyloxy. In specific embodiments, R₁ is hexyloxy. In further or alternative embodiments, R₁ is fluoro. In other embodiments, R₁ is chloro. In other embodiments, R₁ is iodo. In other embodiments, R₁ is bromo. In other embodiments, R₁ is haloalkyl. In other embodiments, R₁ is haloC₁₋₆alkyl. In other embodiments, R₁ is haloC₁₋₃alkyl. In other embodiments, R₁ is haloC₁₋₂alkyl. In specific embodiments, R₁ is monofluoromethyl. In specific embodiments, R₁ is difluoromethyl. In specific embodiments, R₁ is trifluoromethyl.

In some embodiments, R₂ is hydroxy. In some embodiments, R₂ is C₁-C₆alkoxy. In further or additional embodiments, R₂ is C₁-C₃alkoxy. In further or additional embodiments, R₂ is C₁-C₂alkoxy. In specific embodiments, R₂ is methoxy. In specific embodiments, R₂ is ethoxy. In specific embodiments, R₂ is propoxy. In specific embodiments, R₂ is iso-propoxy. In specific embodiments, R₂ is butoxy. In specific embodiments, R₂ is iso-butoxy. In specific embodiments, R₂ is sec-butoxy. In specific embodiments, R₂ is tert-butoxy. In specific embodiments, R₂ is pentyloxy. In specific embodiments, R₂ is hexyloxy.

In some embodiments, R₃, R₄, R₅, and R₆ are independently hydrogen, alkoxy, or alkyl. In some embodiments, R₃, R₄, R₅, and R₆ are independently hydrogen or alkyl. In other embodiments, R₃, R₄, R₅, and R₆ are independently hydrogen.

In some embodiments, R₃ is hydrogen. In some embodiments, R₃ is C₁-C₆alkyl. In other embodiments, R₃ is C₁-C₃alkyl. In other embodiments, R₃ is C₁-C₂alkyl. In specific embodiments, R₃ is methyl. In specific embodiments, R₃ is ethyl. In specific embodiments, R₃ is propyl. In specific embodiments, R₃ is iso-propyl. In specific embodiments, R₃ is butyl. In specific embodiments, R₃ is iso-butyl. In specific embodiments, R₃ is sec-butyl. In specific embodiments, R₃ is tert-butyl. In specific embodiments, R₃ is pentyl. In specific embodiments, R₃ is hexyl. In some embodiments, R₃ is C₁-C₆alkoxy. In further or additional embodiments, R₃ is C₁-C₃alkoxy. In further or additional embodiments, R₃ is C₁-C₂alkoxy. In specific embodiments, R₃ is methoxy. In specific embodiments, R₃ is ethoxy. In specific embodiments, R₃ is propoxy. In further or alternative embodiments, R₃ is fluoro. In other embodiments, R₃ is chloro. In other embodiments, R₃ is iodo. In other embodiments, R₃ is bromo. In other embodiments, R₃ is haloalkyl. In other embodiments, R₃ is haloC₁₋₆alkyl. In other embodiments, R₃ is haloC₁₋₃alkyl. In other embodiments, R₃ is haloC₁₋₂alkyl. In specific embodiments, R₃ is monofluoromethyl. In specific embodiments, R₃ is difluoromethyl. In specific embodiments, R₃ is trifluoromethyl.

In some embodiments, R₄ is hydrogen. In some embodiments, R₄ is C₁-C₆alkyl. In other embodiments, R₄ is C₁-C₃alkyl. In other embodiments, R₄ is C₁-C₂alkyl. In specific embodiments, R₄ is methyl. In specific embodiments, R₄ is ethyl. In specific embodiments, R₄ is propyl. In specific embodiments, R₄ is iso-propyl. In specific embodiments, R₄ is butyl. In specific embodiments, R₄ is iso-butyl. In specific embodiments, R₄ is sec-butyl. In specific embodiments, R₄ is tert-butyl. In specific embodiments, R₄ is pentyl. In specific embodiments, R₄ is hexyl. In some embodiments, R₄ is C₁-C₆alkoxy. In further or additional embodiments, R₄ is C₁-C₃alkoxy. In further or additional embodiments, R₄ is C₁-C₂alkoxy. In specific embodiments, R₄ is methoxy. In specific embodiments, R₄ is ethoxy. In specific embodiments, R₄ is propoxy. In further or alternative embodiments, R₄ is fluoro. In other embodiments, R₄ is chloro. In other embodiments, R₄ is iodo. In other embodiments, R₄ is bromo. In other embodiments, R₄ is haloalkyl. In other embodiments, R₄ is haloC₁₋₆alkyl. In other embodiments, R₄ is haloC₁₋₃alkyl. In other embodiments, R₄ is haloC₁₋₂alkyl. In specific embodiments, R₄ is monofluoromethyl. In specific embodiments, R₄ is difluoromethyl. In specific embodiments, R₄ is trifluoromethyl.

In some embodiments, R₅ is hydrogen. In some embodiments, R₅ is C₁-C₆alkyl. In other embodiments, R₅ is C₁-C₃alkyl. In other embodiments, R₅ is C₁-C₂alkyl. In specific embodiments, R₅ is methyl. In specific embodiments, R₅ is ethyl. In specific embodiments, R₅ is propyl. In specific embodiments, R₅ is iso-propyl. In specific embodiments, R₅ is butyl. In specific embodiments, R₅ is iso-butyl. In specific embodiments, R₅ is sec-butyl. In specific embodiments, R₅ is tert-butyl. In specific embodiments, R₅ is pentyl. In specific embodiments, R₅ is hexyl. In some embodiments, R₅ is C₁-C₆alkoxy. In further or additional embodiments, R₅ is C₁-C₃alkoxy. In further or additional embodiments, R₅ is C₁-C₂alkoxy. In specific embodiments, R₅ is methoxy. In specific embodiments, R₅ is ethoxy. In specific embodiments, R₅ is propoxy. In further or alternative embodiments, R₅ is fluoro. In other embodiments, R₅ is chloro. In other embodiments, R₅ is iodo. In other embodiments, R₅ is bromo. In other embodiments, R₅ is haloalkyl. In other embodiments, R₅ is haloC₁₋₆alkyl. In other embodiments, R₅ is haloC₁₋₃alkyl. In other embodiments, R₅ is haloC₁₋₂alkyl. In specific embodiments, R₅ is monofluoromethyl. In specific embodiments, R₅ is difluoromethyl. In specific embodiments, R₅ is trifluoromethyl.

In some embodiments, R₆ is hydrogen. In some embodiments, R₆ is C₁-C₆alkyl. In other embodiments, R₆ is C₁-C₃alkyl. In other embodiments, R₆ is C₁-C₂alkyl. In specific embodiments, R₆ is methyl. In specific embodiments, R₆ is ethyl. In specific embodiments, R₆ is propyl. In specific embodiments, R₆ is iso-propyl. In specific embodiments, R₆ is butyl. In specific embodiments, R₆ is iso-butyl. In specific embodiments, R₆ is sec-butyl. In specific embodiments, R₆ is tert-butyl. In specific embodiments, R₆ is pentyl. In specific embodiments, R₆ is hexyl. In some embodiments, R₆ is C₁-C₆alkoxy. In further or additional embodiments, R₆ is C₁-C₃alkoxy. In further or additional embodiments, R₆ is C₁-C₂alkoxy. In specific embodiments, R₆ is methoxy. In specific embodiments, R₆ is ethoxy. In specific embodiments, R₆ is propoxy. In further or alternative embodiments, R₆ is fluoro. In other embodiments, R₆ is chloro. In other embodiments, R₆ is iodo. In other embodiments, R₆ is bromo. In other embodiments, R₆ is haloalkyl. In other embodiments, R₆ is haloC₁₋₆alkyl. In other embodiments, R₆ is haloC₁₋₃alkyl. In other embodiments, R₆ is haloC₁₋₂alkyl. In specific embodiments, R₆ is monofluoromethyl. In specific embodiments, R₆ is difluoromethyl. In specific embodiments, R₆ is trifluoromethyl.

In some embodiments, R₇ is C₁-C₆alkyl. In other embodiments, R₇ is C₁-C₃alkyl. In other embodiments, R₇ is C₁-C₂alkyl. In specific embodiments, R₇ is methyl. In specific embodiments, R₇ is ethyl. In specific embodiments, R₇ is propyl. In specific embodiments, R₇ is isopropyl. In alternative embodiments, R₇ is hydrogen. In some embodiments, R₇ is methyl or hydrogen.

In some embodiments, R₉ is hydroxy. In some embodiments, R₉ is C₁-C₆alkoxy. In further or additional embodiments, R₉ is C₁-C₃alkoxy. In further or additional embodiments, R₉ is C₁-C₂alkoxy. In specific embodiments, R₉ is methoxy. In specific embodiments, R₉ is ethoxy. In specific embodiments, R₉ is propoxy. In specific embodiments, R₉ is iso-propoxy. In specific embodiments, R₉ is butoxy. In specific embodiments, R₉ is iso-butoxy. In specific embodiments, R₉ is sec-butoxy. In specific embodiments, R₉ is tert-butoxy. In specific embodiments, R₉ is pentyloxy. In specific embodiments, R₉ is hexyloxy.

In certain embodiments, R₁ is hydroxy or alkoxy; R₂ is hydroxy or alkoxy; R₃, R₄, R₅, and R₆ are independently hydrogen, hydroxy, alkoxy, or alkyl; R₇ is alkyl or hydrogen; and R₉ is hydroxy or alkoxy.

In certain embodiments, R₁ is hydroxy or alkoxy; R₂ is hydroxy or alkoxy; R₃, R₄, R₅, and R₆ are independently hydrogen; R₇ is alkyl or hydrogen; and R₉ is hydroxy.

In some embodiments, R₁ is hydroxy or methoxy; R₂ is hydroxy or methoxy; R₃, R₄, R₅, and R₆ are independently hydrogen, hydroxy, methoxy, methyl; R₇ is methyl or hydrogen; and R₉ is hydroxy or methoxy.

In some embodiments, R₁ is hydroxy or methoxy; R₂ is hydroxy or methoxy; R₃, R₄, R₅, and R₆ are independently hydrogen; R₇ is methyl or hydrogen; and R₉ is hydroxy.

For any and all of the embodiments, substituents are selected from among a subset of the listed alternatives.

Any combination of the groups described above for the various variables is contemplated herein. Throughout the specification, groups and substituents thereof are chosen by one skilled in the field to provide stable moieties and compounds.

In some embodiments, the compound of Formula (II) is selected from the following compounds:

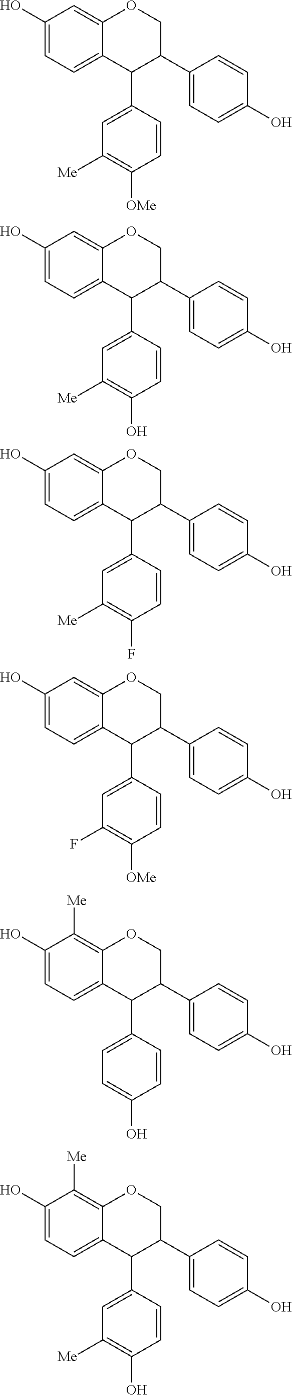

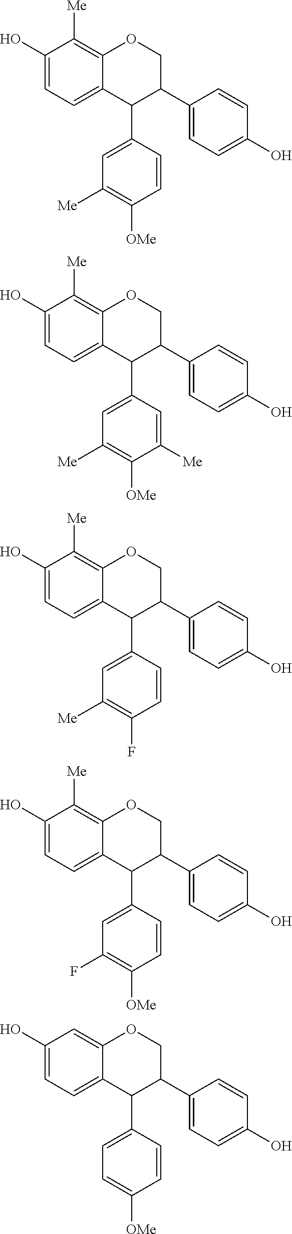

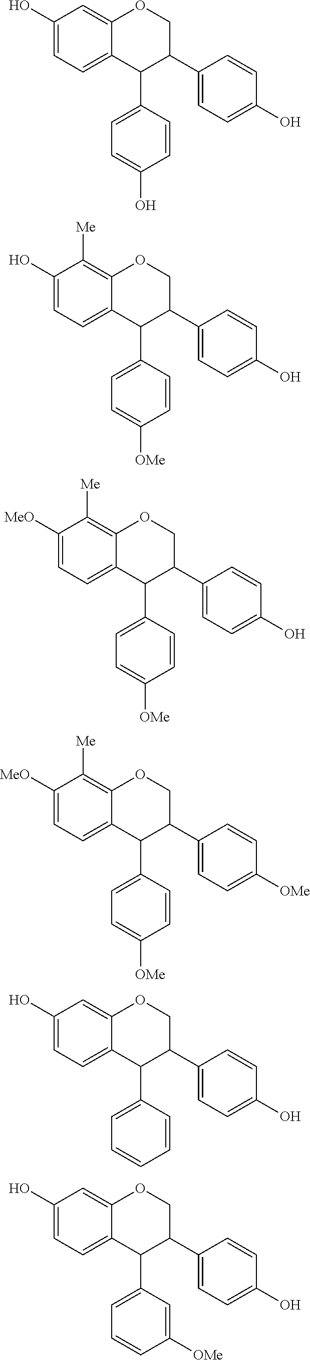

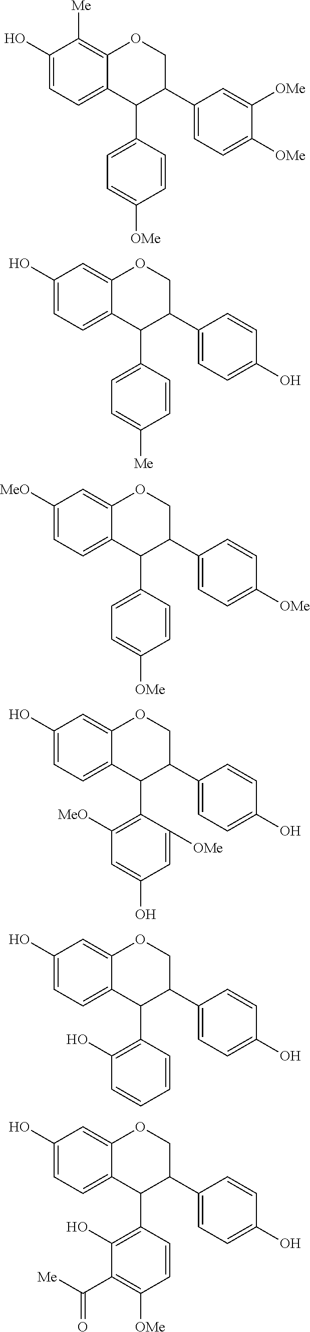

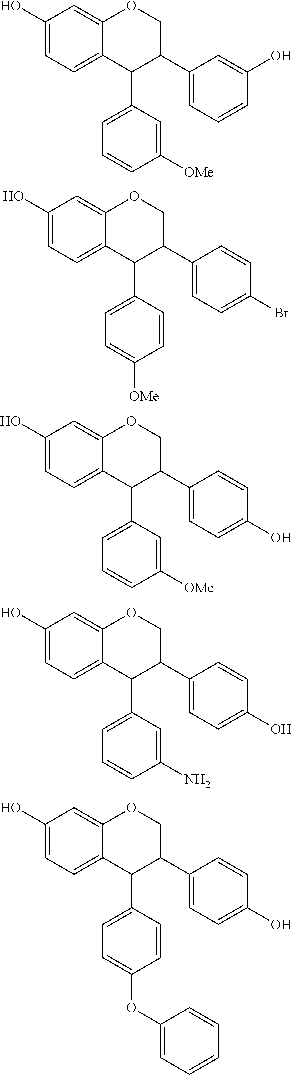

or salts or a derivative thereof.

In some embodiments, the compound of Formula (II) is selected from the following compounds:
3-(4-hydroxyphenyl)-4-(4-methoxy-3-methylphenyl)chroman-7-ol;
3-(4-hydroxyphenyl)-4-(4-hydroxy-3-methylphenyl)chroman-7-ol;
3-(4-hydroxyphenyl)-4-(4-fluoro-3-methylphenyl)chroman-7-ol;
3-(4-hydroxyphenyl)-4-(4-methoxy-3-fluorophenyl)chroman-7-ol;
3-(4-hydroxyphenyl)-4-(4-hydroxyphenyl)-8-methylchroman-7-ol;
3-(4-hydroxyphenyl)-4-(4-hydroxy-3-methylphenyl)-8-methylchroman-7-ol;
3-(4-hydroxyphenyl)-4-(4-methoxy-3-methylphenyl)-8-methylchroman-7-ol;
3-(4-hydroxyphenyl)-4-(4-methoxy-3,5-dimethylphenyl)-8-methylchroman-7-ol;
3-(4-hydroxyphenyl)-4-(4-fluoro-3-methylphenyl)-8-methylchroman-7-ol;
3-(4-hydroxyphenyl)-4-(4-methoxy-3-fluorophenyl)-8-methylchroman-7-ol;
3-(4-hydroxyphenyl)-4-(4-methoxyphenyl)chroman-7-ol;
3-(4-hydroxyphenyl)-4-(4-hydroxyphenyl)chroman-7-ol;
3-(4-hydroxyphenyl)-4-(4-methoxyphenyl)-8-methylchroman-7-ol;
3-(4-hydroxyphenyl)-4-(4-methoxyphenyl)-7-methoxy-8-methylchroman;
3-(4-methoxyphenyl)-4-(4-methoxyphenyl)-7-methoxy-8-methylchroman-7-ol;
3-(4-hydroxyphenyl)-4-phenylchroman-7-ol;
3-(4-hydroxyphenyl)-4-(3-methoxyphenyl)chroman-7-ol;
3-(3,4-dimethoxyphenyl)-4-(4-methoxyphenyl)-8-methylchroman-7-ol;
3-(4-hydroxyphenyl)-4-p-tolylchroman-7-ol;
3-(4-methoxyphenyl)-4-(4-methoxyphenyl)-7-methoxychroman;
4-(4-hydroxy-2,6-dimethoxyphenyl)-3-(4-hydroxyphenyl)chroman-7-ol;
3-(4-hydroxyphenyl)-4-(2-hydroxyphenyl)chroman-7-ol;
3-(4-hydroxyphenyl)-4-(3-acyl-2-hydroxy-4-methoxyphenyl)chroman-7-ol;
3-(3-hydroxyphenyl)-4-(3-methoxyphenyl)chroman-7-ol;
3-(4-bromophenyl)-4-(4-methoxyphenyl)chroman-7-ol;
3-(4-hydroxyphenyl)-4-(3-methoxyphenyl)chroman-7-ol;
4-(3-aminophenyl)-3-(4-hydroxyphenyl)chroman-7-ol; and
3-(4-hydroxyphenyl)-4-(4-phenoxyphenyl)chroman-7-ol.

It will be clear to persons skilled in the art that in the compounds according to certain embodiments of the invention, the aryl substituents on the heterocyclic ring can be cis or trans relative to each other. In certain embodiments of the invention, these substituents will be cis.

In some embodiments, the compound of Formula (II) is selected from the following compounds:

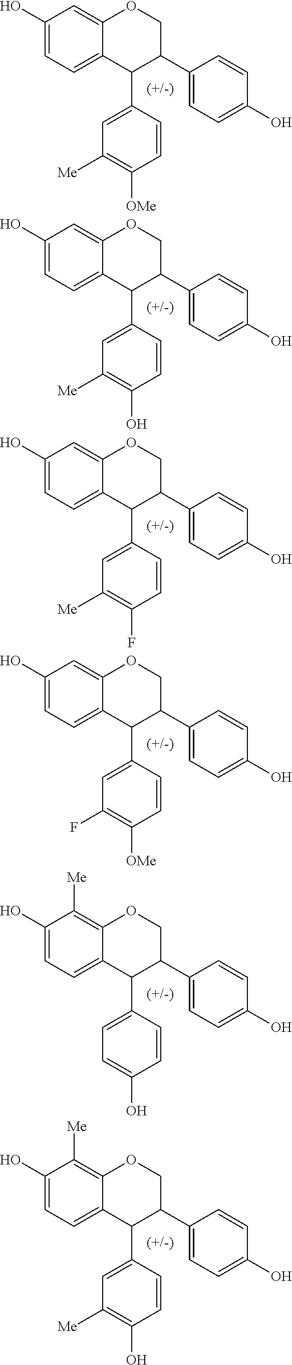

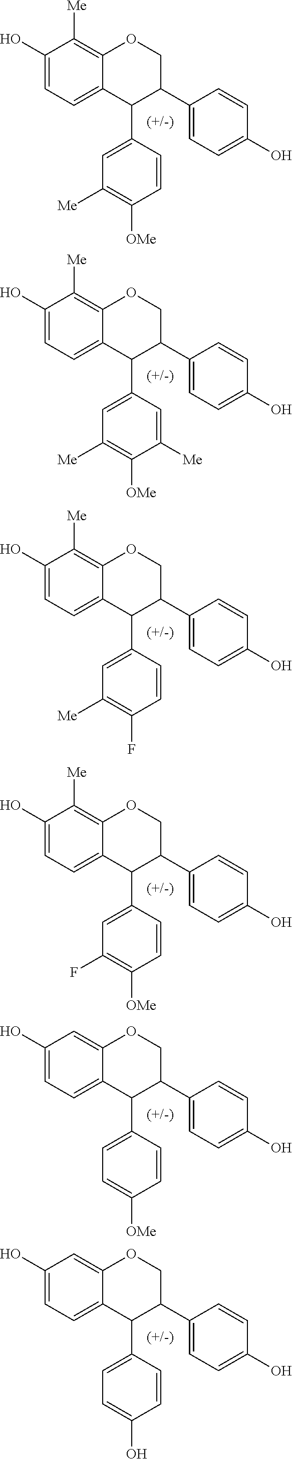

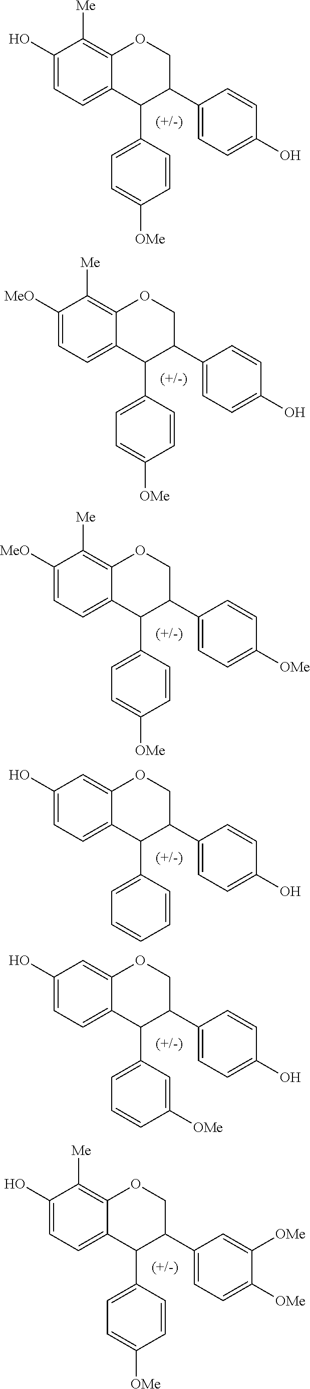

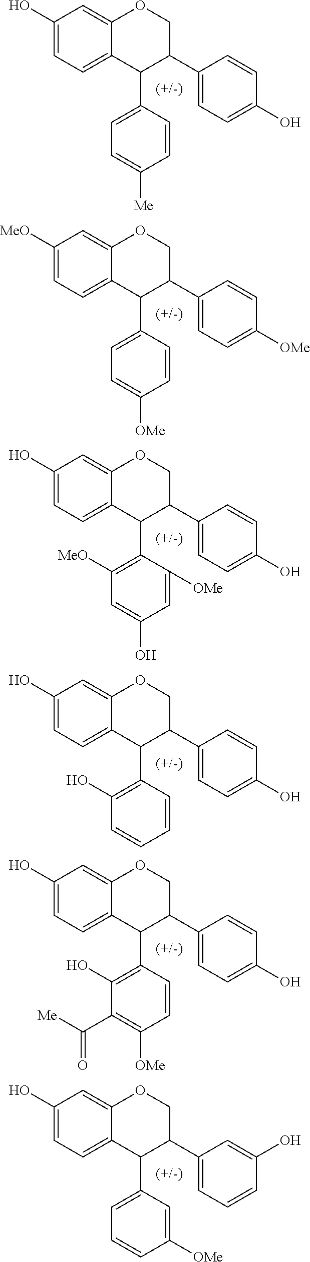

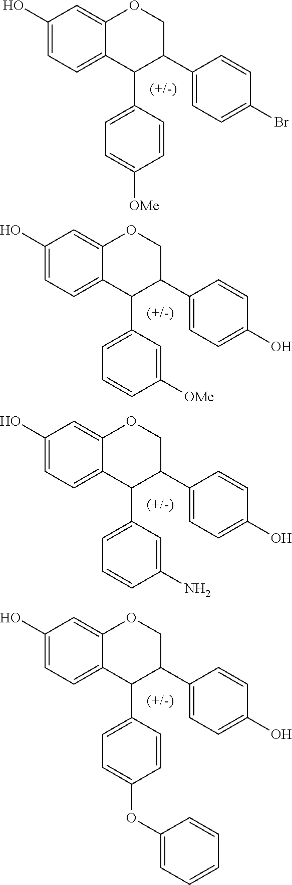

or salts or a derivative thereof; wherein the aryl substituents on the heterocyclic ring are cis relative to each other.

In some embodiments, the compound of Formula (II) is selected from the following compounds:
cis-3-(4-hydroxyphenyl)-4-(4-methoxy-3-methylphenyl)chroman-7-ol;
cis-3-(4-hydroxyphenyl)-4-(4-hydroxy-3-methylphenyl)chroman-7-ol;
cis-3-(4-hydroxyphenyl)-4-(4-fluoro-3-methylphenyl)chroman-7-ol;
cis-3-(4-hydroxyphenyl)-4-(4-methoxy-3-fluorophenyl)chroman-7-ol;
cis-3-(4-hydroxyphenyl)-4-(4-hydroxyphenyl)-8-methylchroman-7-ol;
cis-3-(4-hydroxyphenyl)-4-(4-hydroxy-3-methylphenyl)-8-methylchroman-7-ol;
cis-3-(4-hydroxyphenyl)-4-(4-methoxy-3-methylphenyl)-8-methylchroman-7-ol;
cis-3-(4-hydroxyphenyl)-4-(4-methoxy-3,5-dimethylphenyl)-8-methylchroman-7-ol;
cis-3-(4-hydroxyphenyl)-4-(4-fluoro-3-methylphenyl)-8-methylchroman-7-ol;
cis-3-(4-hydroxyphenyl)-4-(4-methoxy-3-fluorophenyl)-8-methylchroman-7-ol;
cis-3-(4-hydroxyphenyl)-4-(4-methoxyphenyl)chroman-7-ol;
cis-3-(4-hydroxyphenyl)-4-(4-hydroxyphenyl)chroman-7-ol;
cis-3-(4-hydroxyphenyl)-4-(4-methoxyphenyl)-8-methylchroman-7-ol;
cis-3-(4-hydroxyphenyl)-4-(4-methoxyphenyl)-7-methoxy-8-methylchroman;
cis-3-(4-methoxyphenyl)-4-(4-methoxyphenyl)-7-methoxy-8-methylchroman-7-ol;
cis-3-(4-hydroxyphenyl)-4-phenylchroman-7-ol;
cis-3-(4-hydroxyphenyl)-4-(3-methoxyphenyl)chroman-7-ol;
cis-3-(3,4-dimethoxyphenyl)-4-(4-methoxyphenyl)-8-methylchroman-7-ol;
cis-3-(4-hydroxyphenyl)-4-p-tolylchroman-7-ol;
cis-3-(4-methoxyphenyl)-4-(4-methoxyphenyl)-7-methoxychroman;
cis-4-(4-hydroxy-2,6-dimethoxyphenyl)-3-(4-hydroxyphenyl)chroman-7-ol;
cis-3-(4-hydroxyphenyl)-4-(2-hydroxyphenyl)chroman-7-ol;
cis-3-(4-hydroxyphenyl)-4-(3-acyl-2-hydroxy-4-methoxyphenyl)chroman-7-ol;
cis-3-(3-hydroxyphenyl)-4-(3-methoxyphenyl)chroman-7-ol;
cis-3-(4-bromophenyl)-4-(4-methoxyphenyl)chroman-7-ol;
cis-3-(4-hydroxyphenyl)-4-(3-methoxyphenyl)chroman-7-ol;
cis-4-(3-aminophenyl)-3-(4-hydroxyphenyl)chroman-7-ol; and
cis-3-(4-hydroxyphenyl)-4-(4-phenoxyphenyl)chroman-7-ol.

The compounds according to some embodiments of this invention include two chiral centers. The present invention includes all the enantiomers and diastereomers as well as mixtures thereof in any proportions. The invention also extends to isolated enantiomers or pairs of enantiomers. Some of the compounds herein (including, but not limited to benzopyran derivatives and reagents for producing the aforementioned compounds) have asymmetric carbon atoms and can therefore exist as enantiomers or diastereomers. Diastereomeric mixtures can be separated into their individual diastereomers on the basis of their physical chemical differences by methods such as chromatography and/or fractional crystallization. Enantiomers can be separated by converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers, and mixtures thereof are considered as part of the compositions described herein.

The compounds according to some embodiments are racemic mixture. In other embodiments, any compound described herein is in the optically pure form (e.g., optically active (+) and (−), (R)- and (S)-, d- and l-, or (D)- and (L)-isomers). In certain preferred embodiments, the compound of Formula (II) is the d-isomer. Accordingly, provided herein, in some embodiments, is the optically active d-isomer having a structure of Formula (II) in enantiomeric excess. In some embodiments, the d-isomer of the compound of Formula (I), (II), (III), or (IV) is provided in at least 50%, 55%, 60%, 65%, 70%, 75%, 80%, 85%, 88%, 90%, 91%, 92%, 93%, 94%, 95%, 96%, 97%, 98%, 99%, 95%, or 99.9% enantiomeric excess. In other embodiments, the d-isomer of the compound of Formula (II) is provided in greater than 50%, 55%, 60%, 65%, 70%, 75%, 80%, 85%, 88%, 90%, 91%, 92%, 93%, 94%, 95%, 96%, 97%, 98%, 99%, 99.5%, or 99.9% enantiomeric excess. In specific embodiments, the compound of Formula (II) has greater than 90% enantiomeric excess. In specific embodiments, the compound of Formula (II) has greater than 95% enantiomeric excess. In specific embodiments, the compound of Formula (II) has greater than 96% enantiomeric excess. In specific embodiments, the compound of Formula (II) has greater than 97% enantiomeric excess. In specific embodiments, the compound of Formula (II) has greater than 98% enantiomeric excess. In specific embodiments, the compound of Formula (II) has 99% enantiomeric excess or greater. In specific embodiments, the compound of Formula (II) has greater than 99% enantiomeric excess. In specific embodiments, the compound of Formula (II) has 99.5% enantiomeric excess or greater. In specific embodiments, the compound of Formula (II) has greater than 99.5% enantiomeric excess. In specific embodiments, the compound of Formula (II) has 99.8% enantiomeric excess or greater. In specific embodiments, the compound of Formula (II) has greater than 99.8% enantiomeric excess.

In some embodiments, the compound of Formula (II) is selected from the following compounds:

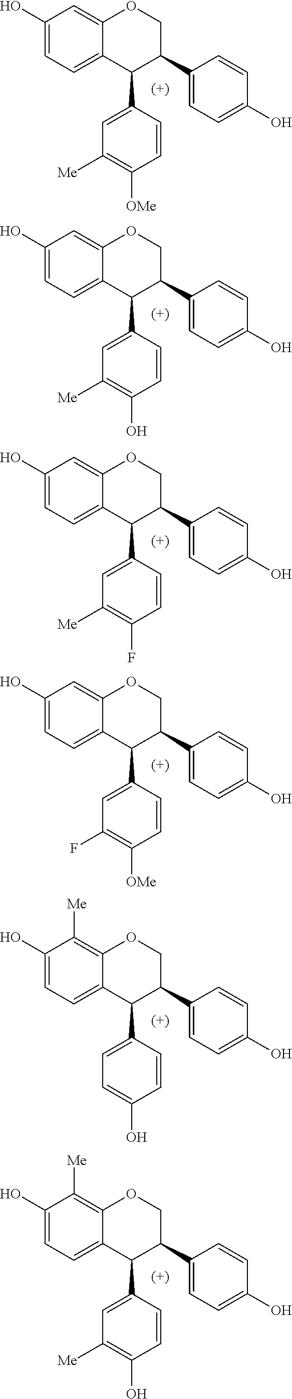

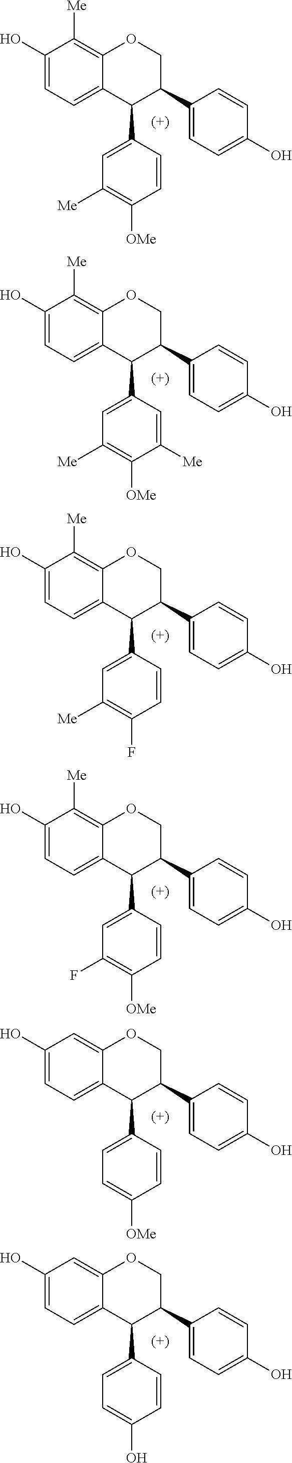

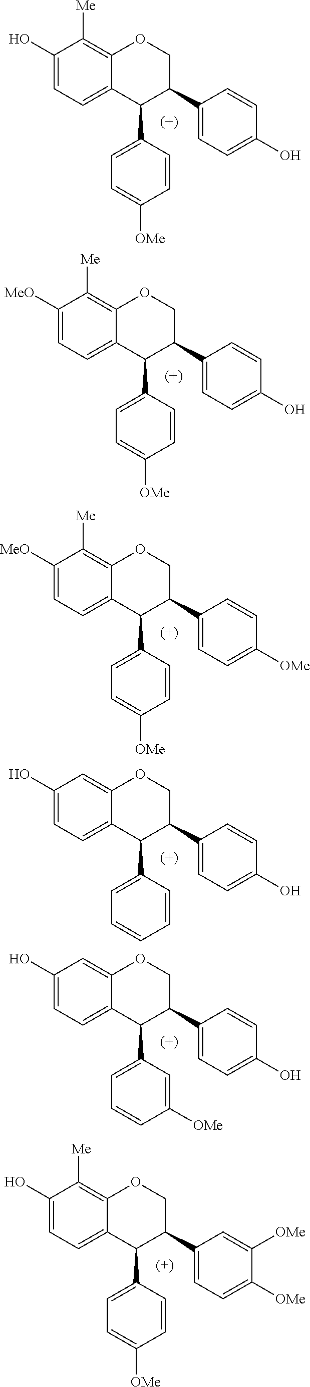

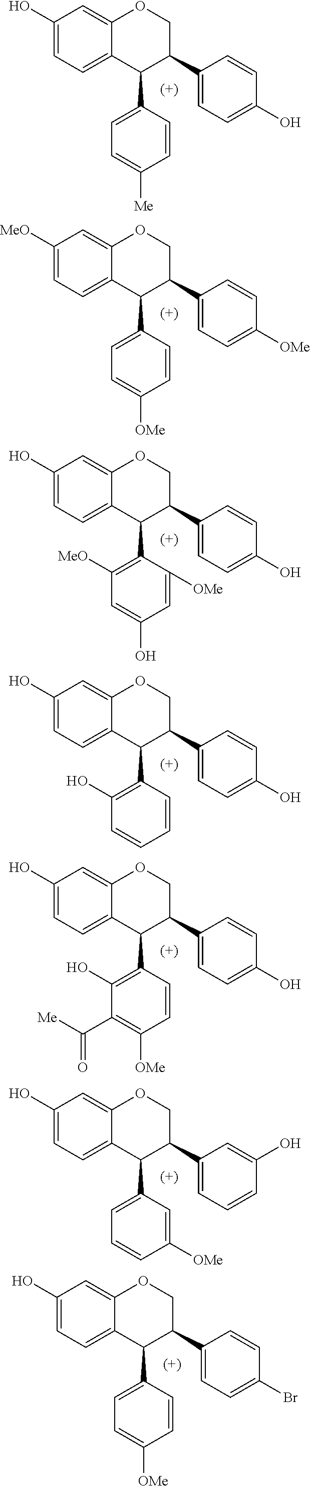

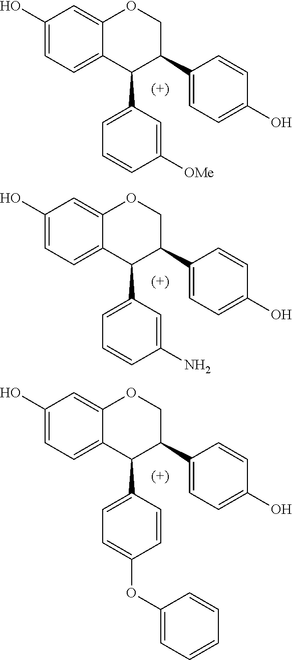

or salts or a derivative thereof.

In some embodiments, the compound of Formula (II) is selected from the following compounds:
d-cis-3-(4-hydroxyphenyl)-4-(4-methoxy-3-methylphenyl)chroman-7-ol;
d-cis-3-(4-hydroxyphenyl)-4-(4-hydroxy-3-methylphenyl)chroman-7-ol;
d-cis-3-(4-hydroxyphenyl)-4-(4-fluoro-3-methylphenyl)chroman-7-ol;
d-cis-3-(4-hydroxyphenyl)-4-(4-methoxy-3-fluorophenyl)chroman-7-ol;
d-cis-3-(4-hydroxyphenyl)-4-(4-hydroxyphenyl)-8-methylchroman-7-ol;
d-cis-3-(4-hydroxyphenyl)-4-(4-hydroxy-3-methylphenyl)-8-methylchroman-7-ol;
d-cis-3-(4-hydroxyphenyl)-4-(4-methoxy-3-methylphenyl)-8-methylchroman-7-ol;
d-cis-3-(4-hydroxyphenyl)-4-(4-methoxy-3,5-dimethylphenyl)-8-methylchroman-7-ol; d-cis-3-(4-hydroxyphenyl)-4-(4-fluoro-3-methylphenyl)-8-methylchroman-7-ol;
d-cis-3-(4-hydroxyphenyl)-4-(4-methoxy-3-fluorophenyl)-8-methylchroman-7-ol;
d-cis-3-(4-hydroxyphenyl)-4-(4-methoxyphenyl)chroman-7-ol;
d-cis-3-(4-hydroxyphenyl)-4-(4-hydroxyphenyl)chroman-7-ol;
d-cis-3-(4-hydroxyphenyl)-4-(4-methoxyphenyl)-8-methylchroman-7-ol;
d-cis-(4-hydroxyphenyl)-4-(4-methoxyphenyl)-7-methoxy-8-methylchroman;
d-cis-(4-methoxyphenyl)-4-(4-methoxyphenyl)-7-methoxy-8-methylchroman-7-ol;
d-cis-3-(4-hydroxyphenyl)-4-phenylchroman-7-ol;
d-cis-3-(4-hydroxyphenyl)-4-(3-methoxyphenyl)chroman-7-ol;
d-cis-3-(3,4-dimethoxyphenyl)-4-(4-methoxyphenyl)-8-methylchroman-7-ol;
d-cis-3-(4-hydroxyphenyl)-4-p-tolylchroman-7-ol;
d-cis-3-(4-methoxyphenyl)-4-(4-methoxyphenyl)-7-methoxychroman;
d-cis-4-(4-hydroxy-2,6-dimethoxyphenyl)-3-(4-hydroxyphenyl)chroman-7-ol;
d-cis-3-(4-hydroxyphenyl)-4-(2-hydroxyphenyl)chroman-7-ol;
d-cis-3-(4-hydroxyphenyl)-4-(3-acyl-2-hydroxy-4-methoxyphenyl)chroman-7-ol;
d-cis-3-(3-hydroxyphenyl)-4-(3-methoxyphenyl)chroman-7-ol;
d-cis-3-(4-bromophenyl)-4-(4-methoxyphenyl)chroman-7-ol;
d-cis-3-(4-hydroxyphenyl)-4-(3-methoxyphenyl)chroman-7-ol;
d-cis-4-(3-aminophenyl)-3-(4-hydroxyphenyl)chroman-7-ol; and
d-cis-3-(4-hydroxyphenyl)-4-(4-phenoxyphenyl)chroman-7-ol.

In some embodiments, the compound of Formula (II) is selected from the following compounds:

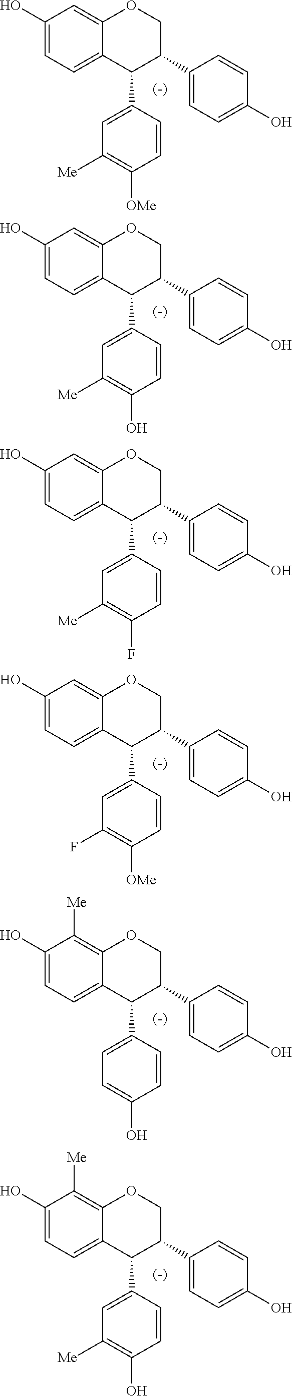

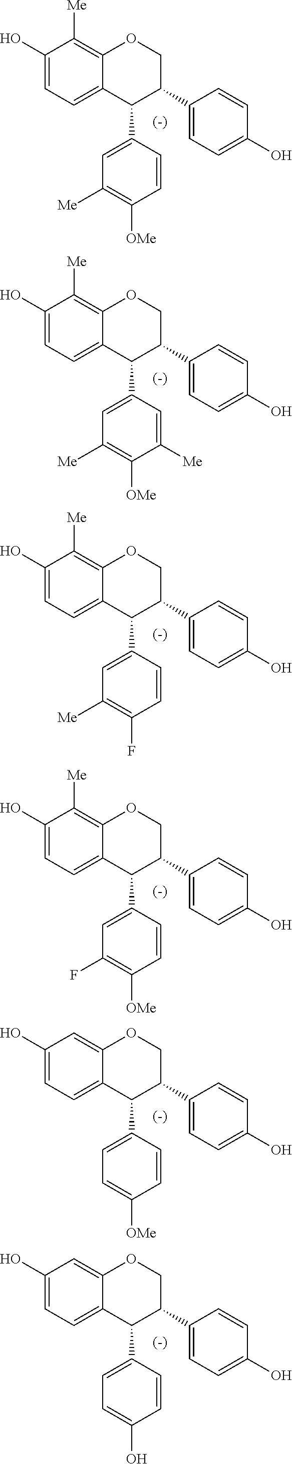

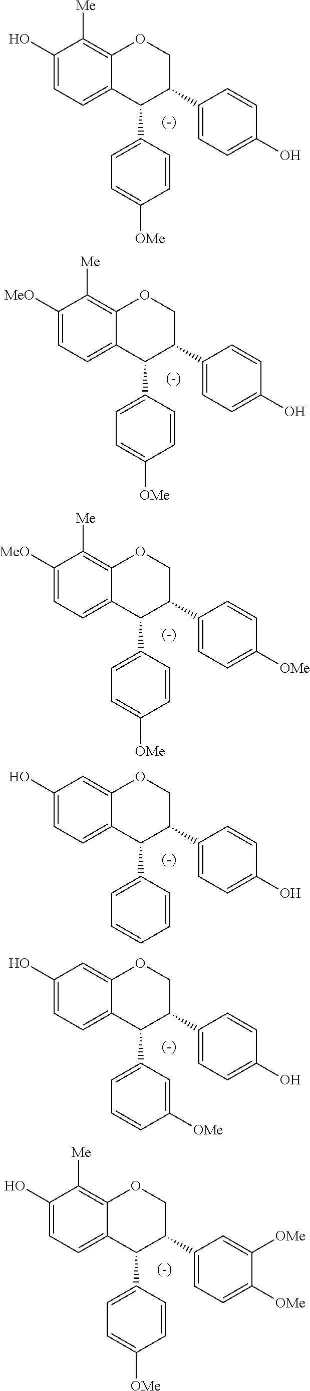

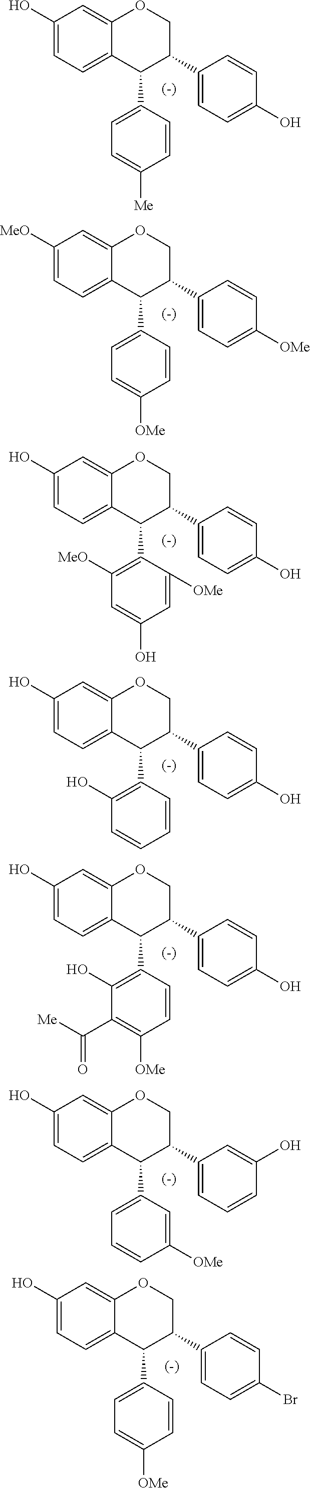

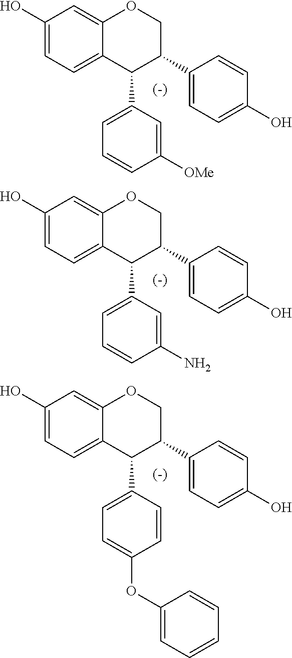

or salts or a derivative thereof.

In some embodiments, the compound of Formula (II) is selected from the following compounds:
l-cis-3-(4-hydroxyphenyl)-4-(4-methoxy-3-methylphenyl)chroman-7-ol;
l-cis-3-(4-hydroxyphenyl)-4-(4-hydroxy-3-methylphenyl)chroman-7-ol;
l-cis-3-(4-hydroxyphenyl)-4-(4-fluoro-3-methylphenyl)chroman-7-ol;
l-cis-3-(4-hydroxyphenyl)-4-(4-methoxy-3-fluorophenyl)chroman-7-ol;
l-cis-3-(4-hydroxyphenyl)-4-(4-hydroxyphenyl)-8-methylchroman-7-ol;
l-cis-3-(4-hydroxyphenyl)-4-(4-hydroxy-3-methylphenyl)-8-methylchroman-7-ol;
l-cis-3-(4-hydroxyphenyl)-4-(4-methoxy-3-methylphenyl)-8-methylchroman-7-ol;
l-cis-3-(4-hydroxyphenyl)-4-(4-methoxy-3,5-dimethylphenyl)-8-methylchroman-7-ol;
l-cis-3-(4-hydroxyphenyl)-4-(4-fluoro-3-methylphenyl)-8-methylchroman-7-ol;
l-cis-3-(4-hydroxyphenyl)-4-(4-methoxy-3-fluorophenyl)-8-methylchroman-7-ol;
l-cis-3-(4-hydroxyphenyl)-4-(4-methoxyphenyl)chroman-7-ol;
l-cis-3-(4-hydroxyphenyl)-4-(4-hydroxyphenyl)chroman-7-ol;
l-cis-3-(4-hydroxyphenyl)-4-(4-methoxyphenyl)-8-methylchroman-7-ol;
l-cis-(4-hydroxyphenyl)-4-(4-methoxyphenyl)-7-methoxy-8-methylchroman;
l-cis-(4-methoxyphenyl)-4-(4-methoxyphenyl)-7-methoxy-8-methylchroman-7-ol;
l-cis-3-(4-hydroxyphenyl)-4-phenylchroman-7-ol;
l-cis-3-(4-hydroxyphenyl)-4-(3-methoxyphenyl)chroman-7-ol;
l-cis-3-(3,4-dimethoxyphenyl)-4-(4-methoxyphenyl)-8-methylchroman-7-ol;
l-cis-3-(4-hydroxyphenyl)-4-p-tolylchroman-7-ol;
l-cis-3-(4-methoxyphenyl)-4-(4-methoxyphenyl)-7-methoxychroman;
l-cis-4-(4-hydroxy-2,6-dimethoxyphenyl)-3-(4-hydroxyphenyl)chroman-7-ol;
l-cis-3-(4-hydroxyphenyl)-4-(2-hydroxyphenyl)chroman-7-ol;
l-cis-3-(4-hydroxyphenyl)-4-(3-acyl-2-hydroxy-4-methoxyphenyl)chroman-7-ol;
l-cis-3-(3-hydroxyphenyl)-4-(3-methoxyphenyl)chroman-7-ol;
l-cis-3-(4-bromophenyl)-4-(4-methoxyphenyl)chroman-7-ol;
l-cis-3-(4-hydroxyphenyl)-4-(3-methoxyphenyl)chroman-7-ol;
l-cis-4-(3-aminophenyl)-3-(4-hydroxyphenyl)chroman-7-ol; and
l-cis-3-(4-hydroxyphenyl)-4-(4-phenoxyphenyl)chroman-7-ol.

In other embodiments, the compound of Formula (II) is 3-(4-hydroxyphenyl)-4-(4-hydroxyphenyl)-8-methylchroman-7-ol:

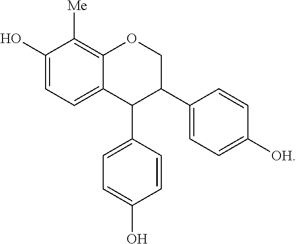 [SMILES: Cc1c(O)ccc2c1OCC(c1ccc(O)cc1)C2c1ccc(O)cc1]

In other embodiments, the compound of Formula (II) is cis-3-(4-hydroxyphenyl)-4-(4-hydroxyphenyl)-8-methylchroman-7-ol:

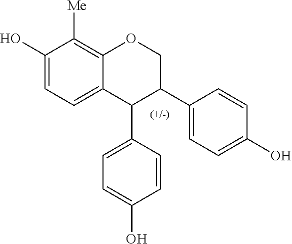 [SMILES: Cc1c(O)ccc2c1OCC(c1ccc(O)cc1)C2c1ccc(O)cc1]

wherein the aryl substituents on the heterocyclic ring are cis relative to one another.

In other embodiments, the compound of Formula (II) is d-cis-3-(4-hydroxyphenyl)-4-(4-hydroxyphenyl)-8-methylchroman-7-ol:

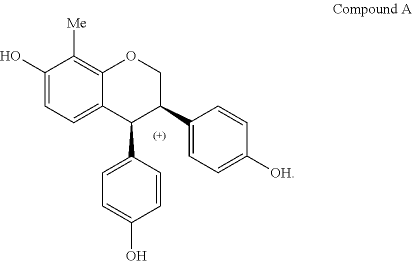 [SMILES: Cc1c(O)ccc2c1OC[C@@H](c1ccc(O)cc1)[C@H]2c1ccc(O)cc1]

In certain embodiments, the compound of Formula (II) is the d-isomer. Accordingly, provided herein, in some embodiments, is the optically active d-isomer having a structure of Formula (II) in enantiomeric excess. In some embodiments, the d-isomer is provided in at least 50%, 55%, 60%, 65%, 70%, 75%, 80%, 85%, 88%, 90%, 91%, 92%, 93%, 94%, 95%, 96%, 97%, 98%, 99%, 95.5%, or 99.9% enantiomeric excess. In other embodiments, the d-isomer is provided in greater than 50%, 55%, 60%, 65%, 70%, 75%, 80%, 85%, 88%, 90%, 91%, 92%, 93%, 94%, 95%, 96%, 97%, 98%, 99%, 99.5%, or 99.9% enantiomeric excess. In some embodiments, the compound of Formula (II) has greater than 90% enantiomeric excess. In specific embodiments, the compound of Formula (II) has greater than 95% enantiomeric excess. In specific embodiments, the compound of Formula (II) has greater than 96% enantiomeric excess. In specific embodiments, the compound of Formula (II) has greater than 97% enantiomeric excess. In specific embodiments, the compound of Formula (II) has greater than 98% enantiomeric excess. In specific embodiments, the compound of Formula (II) has greater than 99% enantiomeric excess. In specific embodiments, the compound of Formula (II) has greater than 99.9% enantiomeric excess.

In additional or further embodiments, the compounds described herein are used in the form of pro-drugs. In additional or further embodiments, the compounds described herein are metabolized upon administration to an organism in need to produce a metabolite that is then used to produce a desired effect, including a desired therapeutic effect.

Any compound described herein may be synthesized according to the exemplary synthesis shown in Scheme 1.

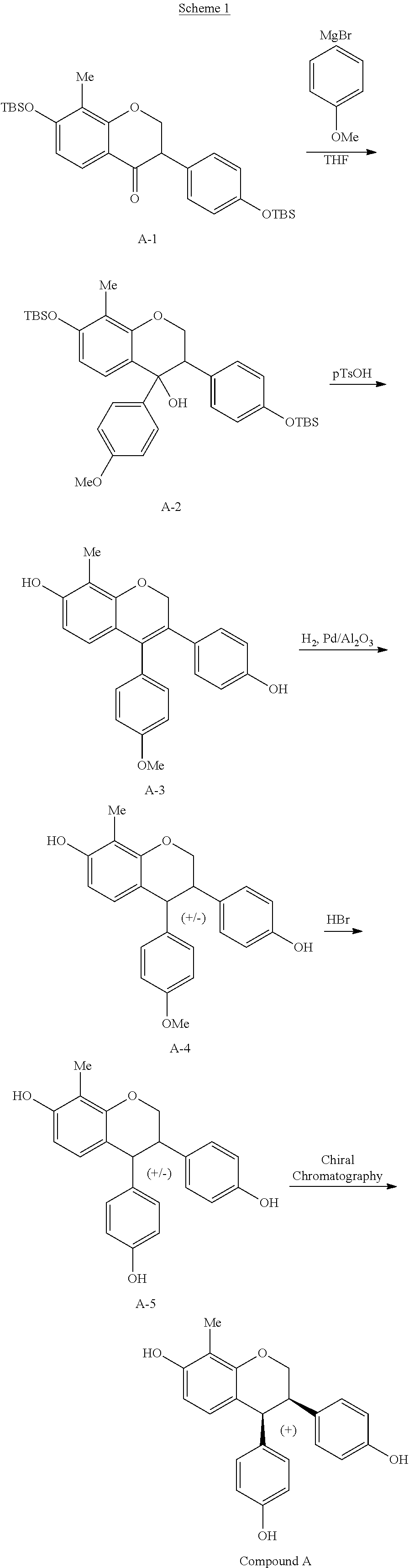

Intermediate A-1, the synthesis of which can be found in WO2006/032085, is added to a 2-neck round bottom flask and flushed under nitrogen. Anhydrous THF is added and a condenser is attached to the reaction vessel, which is then cooled to 0° C. Commercial 4-methoxyphenylmagnesium bromide (0.5 M solution in THF) is added to the reaction mixture dropwise over 10 minutes. The reaction is quenched by the dropwise addition of wet ether under nitrogen, with a white precipitate forming as quenching proceeds. Additional water is added to the reaction mixture before extracting with diethyl ether. The organic layers are combined and washed with water and brine, then dried over anhydrous magnesium sulfate. Solvent is removed in vacuo to yield intermediate A-2 as a clear yellow oil which solidifies to an off-white solid overnight.

Intermediate A-2 (4.2 g), para-toluene sulphonic acid (pTsOH), boiling chips (4.5 g) and ethanol (200 mL) are combined in a 2-neck 500 mL round bottom flask with condenser attached. The reaction is heated at reflux for 3 hours before concentrating in vacuo to ˜20 mL. The reaction mixture is poured into chilled, stirred water (˜100 mL). The mixture is then extracted with ethyl acetate, and the combined organic layers are washed with water (3×100 mL), then brine (1×100 mL), then dried over anhydrous magnesium sulfate and filtered. Solvent is removed in vacuo. The residual oil is purified by recrystallization in methanol (15 mL), providing intermediate A-3.

Intermediate A-4 can be prepared from intermediate A-3 (2.5 g), 10% Pd/Al₂O₃ (0.4 g) and ethanol (50 mL). Reagents are combined in a 2-neck 100 mL round bottom flask, and the reaction is hydrogenated at low pressure using standard conditions for 3 hours. The reaction is filtered through Celite to remove the catalyst, then rinsed through with ethanol (100 mL). The filtrate is concentrated to −15 mL before being poured into chilled, stirred water (˜300 mL). A pale orange precipitate forms which then comes a brown oil. The mixture is extracted with diethyl ether, and the combined organic layers are washed with water (3×100 mL), then brine (1×100 mL), then dried over anhydrous magnesium sulfate and filtered. The solvent is removed in vacuo to give red-brown oil. The product is recrystallised from diethyl ether (˜15 mL), to give a brown solid, which is further rinsed with chilled diethyl ether to give intermediate A-4.

Isolated intermediate A-4 is transferred to a flask purged with nitrogen. Hydrogen bromide in acetic acid (33 wt %) is added drop-wise to the reaction mixture. The mixture is heated to reflux at 130° C. for 7 h. The reaction mixture is placed in an ice bath and the pH is adjusted to 6. The reaction mixture is extracted with EtOAc and the organic layer is washed with water, brine, dried over magnesium sulfate, filtered and concentrated in vacuo. The resultant residue is purified by column chromatography to yield intermediate A-5. Compound A is isolated from intermediate A-5 by chiral chromatography following known methods. In some embodiments, Compound A is isolated at 99% enantiomeric excess.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound described herein as set forth herein are included within the scope of the claims. In some cases, some of the herein-described compounds are prodrugs for another derivative or active compound.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

### Bcl-2 Family Inhibitors

Some embodiments provided herein describe Bcl-2 family inhibitors that are capable of inhibiting at least one member of the family of Bcl-2 proteins. In some embodiments, the Bcl-2 family inhibitor is docetaxel, venetoclax, navitoclax, sabutoclax, obatoclax, apoptone, isosorbide, rasagiline, eribulin, dexibuprofen, glycine betaine, ABT-263, ABT-737, APG 2575, APG 1252, AT-101, G3139 (genasense or oblimersen), HA14-1, TW-37, antimycinA, apogossypol, 544563 or a pharmaceutically acceptable salt thereof. In some embodiments, the Bcl-2 family inhibitor is venetoclax, navitoclax, obatoclax mesylate, ABT-737, APG 2575, APG 1252, AT-101, or a pharmaceutically acceptable salt thereof. In some embodiments, the Bcl-2 family inhibitor is venetoclax. In some embodiments, the Bcl-2 family inhibitor interacts with a Bcl-2 family protein. In some embodiments, the Bcl-2 family inhibitor interacts with a caspase. In some embodiments, the Bcl-2 family inhibitor interacts with mitochondrial outer membrane permeabilization (MOMP). In some embodiments, the Bcl-2 family inhibitor interacts with BAX, BAK, or a combination of the two. In some embodiments, the Bcl-2 inhibitor interacts with the BH1 domain, BH2 domain, BH3 domain, BH4 domain, or any combination thereof. In some embodiments, the Bcl-2 inhibitor mimics the BH1 domain, BH2 domain, BH3 domain, BH4 domain, or a combination thereof. In some embodiments, the Bcl-2 family inhibitor mimics the BH3 domain. In some embodiments, the Bcl-2 family inhibitor is selective for one protein within the Bcl-2 family. In a preferred embodiment, the Bcl-2 family inhibitor is selective for Bcl-2 protein over other Bcl-2 family proteins. In other embodiments, the Bcl-2 family inhibitor is selective for a subset of proteins within the Bcl-2 family (e.g., antiapoptotic proteins). In yet other embodiments, the Bcl-2 family inhibitor is non-selective. In some embodiments, the Bcl-2 family inhibitor interacts with a Bcl-2 family protein and a non-Bcl-2 family protein. In some embodiments, the Bcl-2 family inhibitor inhibits Bcl-2 family proteins through a signaling partner such as AMRA1, APR, B2L11-2, B2L11, B2L13, B2L14, B2CL2, B2L10, B2CL1, BAD, CISD2, BECN1, BIK, BIM, P53, B2L11-1, BBC3, BAX, BAP31, BRCA1, SIVA, NLRP1, LRRK2, BAK, NR4A1, ITPR1, BCLF1, BMF, BAD, ASPP2, BID, BNI3L, MDM4, RAF1, EGLN3, or any combination thereof. In some embodiments, the Bcl-2 family inhibitor inhibits a protein-protein interaction. In some embodiments, the protein-protein interaction interrupted by the Bcl-2 family inhibitor is a Bcl-2/Bcl-2 dimer, a Bcl-2/BAD complex, a Bcl-2/BID complex, a Bcl-2/PUMA complex, a Bcl-2/BIM complex, or a combination thereof. In a preferred embodiment, the protein-protein interaction inhibited by the Bcl-2 family inhibitor is a Bcl-2/BIM interaction. In some embodiments, the Bcl-2 family inhibitor is an allosteric modulator. In some embodiments, the Bcl-2 family inhibitor is a covalent inhibitor. In some embodiments, the Bcl-2 family inhibitor mimics a peptide (e.g., peptidomimetic). In specific instances, the Bcl-2 family inhibitor mimics a BH3 domain. In some embodiments, the Bcl-2 family inhibitor is an orthosteric ligand. In some embodiments, the Bcl-2 family inhibitor mimics an endogenous ligand.

In some embodiments, the Bcl-2 family protein is an anti-apoptotic protein, a pro-apoptotic pore-former, a pro-apoptotic BH3-only protein, or any combination thereof. In some embodiments, the Bcl-2 family protein is Bcl-1, Bcl-2, Bcl-b, Bcl-x, Bcl-xL, Bcl-w, Bcl-g, Bcl-RAMBO, MCL-1, BNIP-3, BFL-1/A1, BAX, BAK, BOK, BAD, BID, BIK, BIM, BMF, HRK, NOXA, PUMA, BAP31, BECLIN-1, BFK, BOK, SPIKE, BBC3, B2L13, B2L14, B2CL2, B2L10, B2L11, B2CL1, B2LA1, B2L12, or a combination thereof.

In some embodiments, the Bcl-2 family inhibitor interacts with (e.g., inhibits) an alternate form of a Bcl-2 family protein or signaling partner of a Bcl-2 family protein. In some embodiments, alternate forms include mutations, resistance mutations, variant protein splices, homologs, isoforms, fragments, dimers, complexes, domain translocations, or any combination thereof. In some embodiments, the Bcl-2 family protein is resistant to one or more chemotherapeutic agents. In some embodiments, the Bcl-2 family protein is resistant to Bcl-2 family inhibitors. In some embodiments, the Bcl-2 family protein is resistant to an oxidative phosphorylation inhibitor (e.g., a compound of Formula (II)). In some embodiments, the inhibition of the Bcl-2 family protein is synergistic with inhibition of oxidative phosphorylation. In some embodiments, the inhibition of the Bcl-2 family protein is additive with inhibition of oxidative phosphorylation. In some embodiments, the Bcl-2 family inhibitor reduces BIM interactions (e.g., with Bcl-2, Mcl-1) to facilitate apoptosis in cancer cells.

In some embodiments, the Bcl-2 family protein is a mitochondrial protein. In some embodiments, the Bcl-2 family protein is nuclear. In some embodiments, the Bcl-2 family protein is localized in the endoplasmic reticulum. In some embodiments, the Bcl-2 family protein is B-cell lymphoma 2 protein (Bcl-2).

### Methods

Some embodiments provided herein describe a method of treating cancer in an individual in need of cancer therapy. In specific embodiments, the methods comprise contacting the cancer or cancer cell with an oxidative phosphorylation inhibitor (e.g., a benzopyran derivative) and a Bcl-2 family inhibitor. In certain embodiments, the cancer or cancer cell is present in an individual. In specific embodiments, the individual is in need of cancer therapy.

In other embodiments, provided herein is a method of treating a disease or disorder associated with dysregulation of cell proliferation. In some embodiments, the disease or disorder is cancer. In some embodiments, the disease or disorder is characterized by an overexpression of Bcl-2 family proteins compared to normal cells. In some embodiments, the disease or disorder is a cancer characterized by an overexpression of Bcl-2 family proteins compared to normal cells. In other embodiments, provided herein is a method of increasing, inducing, or restoring sensitivity to a cancer therapy in an individual. Some embodiments provided herein describe a method of treating a chemoresistant cancer. In specific embodiments, the methods comprise contacting the cancer or cancer cell with an oxidative phosphorylation inhibitor (e.g., a benzopyran derivative of Formula (II)) and a Bcl-2 family inhibitor. In certain embodiments, the cancer or cancer cell is present in an individual. In specific embodiments, the individual is in need of cancer therapy.

In some embodiments, the cancer or cancer cell has lost sensitivity to a chemotherapeutic agent, anti-cancer agent or radiation therapy. In some embodiments, the cancer is resistant to a chemotherapeutic agent (e.g., an oxidative phosphorylation inhibitor) or is “chemoresistant.” In some embodiments, the cancer is resistant to standard of care (“SOC”). In some embodiments, SOC is induction therapy with cytarabine and an anthracycline. In some embodiments, the cancer is resistant to an oxidative phosphorylation inhibitor, a nucleoside chemotherapeutic, a Bcl-2 family inhibitor, or a combination thereof. In some embodiments, the cancer is resistant to an oxidative phosphorylation inhibitor. In some embodiments, the cancer is resistant to cytarabine or azacytidine. In some embodiments, the cancer is resistant to cytarabine (ara-C). In some embodiments, the cancer is resistant to azacytidine. In some embodiments, the cancer is resistant to multiple chemotherapeutic agents (e.g., a Bcl-2 inhibitor and a nucleoside analog). In some embodiments, the cancer is resistant to venetoclax and azacytidine, or venetoclax and cytarabine. In some embodiments the cancer or cancer cell is not resistant or has not lost sensitivity to a chemotherapeutic agent, anti-cancer agent or radiation therapy.

In some embodiments, the combination of an oxidative phosphorylation inhibitor (e.g., a benzopyran derivative) and a Bcl-2 family inhibitor has an enhanced effect. In other embodiments, the combination of an oxidative phosphorylation inhibitor (e.g., a benzopyran derivative), a Bcl-2 family inhibitor, and an additional anti-cancer agent has an enhanced effect. In some embodiments, the combination therapy of a Bcl-2 family inhibitor described herein and a benzopyran derivative (e.g., a compound of Formula (II)) provides a synergistic effect. In some embodiments, the combination therapy of a Bcl-2 family inhibitor described herein and a benzopyran derivative (e.g., a compound of Formula (II)) provides a synergistic antitumor or anti-cancer activity. In some embodiments, the synergistic effect observed with the combination therapy described herein results in improved efficacy of therapies in the prevention, management, treatment, or amelioration of a cancer (e.g., a leukemia or a lung cancer). In some embodiments, the combination therapies and/or compositions described herein chemosensitize cancer cells, wherein the combination therapies and/or compositions lower the amount of anti-cancer agent that is required to kill the cancer cell. In other embodiments, the combination therapies and/or compositions described herein chemosensitize cancer cells, wherein the combination therapies and/or compositions convert cancer cells from a state of chemo-resistant to chemo-sensitive. In further or additional embodiments, the combination therapies and/or compositions described herein radiosensitize cancer cells, wherein the combination therapies and/or compositions lower the amount of gamma-irradiation that is required to kill the cancer cell. In other embodiments, the combination therapies and/or compositions described herein radiosensitize cancer cells, wherein the combination therapies and/or compositions convert cancer cells from a state of radio-resistant to radio-sensitive.

In some embodiments, the combination therapies, treatments, compositions, methods, and kits described herein offer mechanistic advantages to treating cancer compared to chemotherapeutic agents administered alone. In some embodiments, combined inhibition of Bcl-2 family proteins and oxidative phosphorylation inhibitor prevents, reduces, inhibits, reverses, or otherwise negates chemo-resistance in a cell (e.g. a cancer cell). In other embodiments, combined inhibition of Bcl-2 protein and oxidative phosphorylation inhibitor reduces Bcl-2 protein mediated resistance to oxidative phosphorylation inhibitors. In yet other embodiments, combined inhibition of Bcl-2 family proteins and oxidative phosphorylation inhibitor reduces Bcl-xL protein mediated resistance to oxidative phosphorylation inhibitors. In some embodiments, the combination therapies disclosed herein reduce chemo-resistance. In other embodiments, the combination therapies disclosed herein enhance chemotherapeutic benefit (e.g., enhanced apoptosis or necrosis) in non-chemo-resistant cells (e.g., cancer cells). In preferred embodiments, the combination therapy induces apoptosis, necrosis, or other cell death pathways in cancerous cells. In some embodiments, therapeutic effects (e.g., induction of cell death in cancer cells, reduction in proliferation or survival of cancer cells) is higher for the combination therapy than either agent alone.

In some embodiments, the combination therapies disclosed herein reduce the ability of Bcl-2 family proteins to increase oxidative phosphorylation. In other embodiments, a combination therapy reduces a Bcl-2 family protein's ability to interact with a cell's mitochondrial function. In other embodiments, a combination therapy disclosed herein at least partially restores or enhances the efficacy of an anti-cancer agent in treating cancer. In some embodiments, inhibition of a Bcl-2 family protein results in reduced oxidative phosphorylation. In some embodiments, a combination disclosed herein synergistically reduces oxidative phosphorylation, thereby reducing cancer cell survival.

In some embodiments, the cancer is drug-resistant or chemoresistant. In some embodiments, the cancer is multi-drug resistant. As used herein, a “drug-resistant cancer” is a cancer that is resistant to conventional commonly known cancer therapies. Examples of conventional cancer therapies include treatment of the cancer with agents such as methotrexate, doxorubicin, 5-fluorouracil, vincristine, vinblastine, pamidronate disodium, anastrozole, exemestane, cyclophosphamide, epirubicin, toremifene, letrozole, trastuzumab, megestrol, tamoxifen, paclitaxel, docetaxel, capecitabine, goserelin acetate, etc. A “multi-drug resistant cancer” is a cancer that resists more than one type or class of cancer agents, i.e., the cancer is able to resist a first drug having a first mechanism of action, and a second drug having a second mechanism of action.

In some embodiments, a cancer cell (e.g., a leukemia stem cell) is dependent on oxidative phosphorylation for energy production. In some embodiments, standard of care (“SOC”) treatment regimens, e.g., standard induction chemotherapy, does not eliminate oxidative phosphorylation dependent cancer cells. In some embodiments, the combination of an oxidative phosphorylation inhibitor (e.g., a benzopyran derivative of Formula II) with a Bcl-2 inhibitor (e.g., venetoclax) induces cell death in a cancer cell reliant on mitochondrial metabolism. In some embodiments, the combination of an oxidative phosphorylation inhibitor (e.g., a benzopyran derivative of Formula II) with a Bcl-2 inhibitor (e.g., venetoclax) is effective in treating cancers that are resistant or non-responsive to SOC therapy. In some embodiments, SOC therapy comprises a nucleoside analog (e.g., cytarabine, gemcitabine, and the like), and an anthracycline drug (e.g., daunorubicin, idarubicin, and the like).

In some embodiments, the cancer is resistant or non-responsive to a nucleoside analog and an anthracycline drug. In some embodiments, the cancer is resistant or non-responsive to cytarabine and an anthracycline drug. In some embodiments, the cancer is resistant or non-responsive to cytarabine and daunorubicin. In some embodiments, the cancer is resistant or non-responsive to cytarabine and idarubicin. In some embodiments, the combination of an oxidative phosphorylation inhibitor (e.g., a benzopyran derivative of Formula II) with a Bcl-2 inhibitor (e.g., venetoclax) is provided in combination with SOC therapy.

Provided herein in some embodiments, is a method to treat cancer in an individual, the method comprising administering to the individual an oxidative phosphorylation inhibitor (e.g., a benzopyran derivative) and a Bcl-2 family inhibitor, wherein the side-effects associated with chemotherapy, radiotherapy, or cancer therapy is reduced or minimized. In some instances, the combination therapies and/or compositions described herein provide chemo-protective and/or radio-protective properties to non-cancerous cells. In further or additional embodiments, the lower amount of oxidative phosphorylation inhibitor (e.g., a benzopyran derivative), a Bcl-2 family inhibitor, or additional anti-cancer agent reduces or minimizes any undesired side-effects associated with chemotherapy. Non-limiting examples of side-effects associated with chemotherapy, radiotherapy or cancer therapy include fatigue, anemia, appetite changes, bleeding problems, diarrhea, constipation, hair loss, nausea, vomiting, pain, peripheral neuropathy, swelling, skin and nail changes, urinary and bladder changes, and trouble swallowing. In some embodiments, the combination therapy provides enhanced patient compliance or tolerability of treatment. In some embodiments, the combination therapy provides reduced toxicity relative to an effective dose of monotherapy. In some embodiments, the incidence of adverse effects is decreased in patients receiving a combination therapy as described herein, compared to standard of care therapy or other monotherapy.

In some instances, cancer cells overexpress Bcl-2 family proteins (e.g., Bcl-2, Bcl-xL) as a mechanism to evade mitochondrial inhibitors (e.g., oxidative phosphorylation inhibitors). Bcl-2 family proteins may enhance a cell's ability to generate energy from oxidative phosphorylation alone. In some instances where oxidative phosphorylation is enhanced by overexpression of Bcl-2, a combination therapy as disclosed herein may be particularly effective at blocking cellular metabolism and thus at killing cancer cells. In one example, AML stem cells are highly dependent on oxidative phosphorylation for survival, and are unable to upregulate glycolysis sufficiently after oxidative phosphorylation is inhibited. Therefore, in some cases, AML stem cells are particularly susceptible to an oxidative phosphorylation inhibitor as described herein. In additional instances, AML cells (e.g., stem cells) overexpress anti-apoptotic Bcl-2 protein(s), making a synergistic combination of an oxidative phosphorylation inhibitor with a Bcl-2 inhibitor as described herein uniquely effective in disrupting chemoresistant AML cells. In some embodiments, this unique metabolic and mitochondrial biology makes chemoresistant AML vulnerable to strategies that target oxidative phosphorylation and Bcl-2.

In some cases, it is advantageous to administer a Bcl-2 family inhibitor concurrent with an oxidative phosphorylation inhibitor. In other cases, a Bcl-2 family inhibitor and an oxidative phosphorylation inhibitor are not administered concurrently. In some embodiments, administration of a Bcl-2 family inhibitor precedes treatment with an oxidative phosphorylation inhibitor. In other embodiments, treatment with an oxidative phosphorylation inhibitor precedes treatment with a Bcl-2 family inhibitor. In some embodiments, a combination therapy as disclosed herein is effective at a lower dose than either agent alone. In additional embodiments, side-effects of chemotherapy may be reduced by reducing the effective amount of a chemotherapeutic agent needed to treat a cancer.

In some embodiments, the cancer is selected from the group consisting of leukemia, lung cancer (both small cell and non-small cell), squamous non-small cell lung cancer, non-squamous non-small cell lung cancer, Lewis lung carcinoma, non-Hodgkin lymphoma, and myeloma. In some embodiments, the cancer is selected from, by way of non-limiting example, leukemia or lung cancer. In some embodiments, the cancer is childhood leukemia, acute myeloid leukemia (AML), chronic myeloid leukemia (CML), acute lymphocytic leukemia (ALL), chronic lymphocytic leukemia (CLL), hairy cell leukemia (HCL), myelodysplastic syndrome (MDS), or a combination thereof. In a specific example, the cancer is acute myeloid leukemia (AML). In some embodiments, the cancer is refractory or relapsed acute myeloid leukemia (r/r AML). In some embodiments, the AML or r/r AML is of the myeloblastic (e.g., M0, M1, or M2), promyelocytic (M3), myelomonocytic (M4), monocytic *M5), erythroleukemia (M6), or megakaryocytic (M7) type. In some embodiments, the AML is of the acute monocytic leukemia (AMoL) type. In some embodiments, the r/r AML is of the acute monocytic leukemia (r/r AMoL) type. In some embodiments, the r/r AMoL is resistant to cytarabine. In some embodiments, the cancer is a non-Hodgkin lymphoma, B-cell lymphoma, diffuse large B-cell lymphoma, small lymphocytic lymphoma (SLL), mantle cell lymphoma, or follicular lymphoma. In other embodiments, the lung cancer is small cell lung cancer (SCLC), non-small cell lung cancer (NSCLC), adenocarcinoma of the lung, squamous cell carcinoma, large-cell undifferentiated carcinoma, metastatic lung cancer, adenosquamous carcinoma of the lung, large cell neuroendocrine carcinoma, salivary gland-type lung carcinoma, lung carcinoids, mesothelioma, sarcomatoid carcinoma of the lung, malignant granular cell lung tumors, or a combination thereof. In some embodiments, the cancer is colorectal cancer, renal cell cancer, hepatic cancer, gastric or gastroesophageal junction adenocarcinoma, gastrointestinal stromal tumor (GIST), thyroid cancer, non-squamous lung cancer, ovarian cancer, cervical cancer, primitive neuro-ectodermal tumors (pNET), and glioblastoma. In some embodiments, the cancer is colorectal cancer or renal cell cancer. A cancer to be treated by use of a composition, method, or kit as disclosed herein may be, by way of non-limiting example, Stage I, Stage II, Stage III, Stage IV, limited stage, extensive stage.

A tumor cell in a subject or individual may be part of any type of cancer as described herein. In some embodiments, the methods described herein are useful in treating various cancers including but not limited childhood leukemia, acute lymphocytic leukemia, acute myeloid leukemia, chronic lymphocytic leukemia, chronic myeloid leukemia, hairy cell leukemia, acute promyelocytic leukemia, plasma cell leukemia, erythroleukemia, myelomas, haematological disorders including myelodysplasia syndromes, myeloproliferative disorders, aplastic anemia, Fanconi anemia, Waldenstroms Macroglobulinemia, Richter syndrome, blastic plasmacytoid dendritic cell neoplasm (BPDCN), multiple myeloma, plasma cell myeloma, diffuse large B-cell lymphoma, small lymphocytic lymphoma (SLL), mantle cell lymphoma, or follicular lymphoma, Hodgkin's disease, non-Hodgkin's lymphoma, cutaneous T-cell lymphoma, peripheral T-cell lymphoma, mature T-cell and NK-cell non-Hodgkin lymphoma, AIDS related Lymphoma, B-cell lymphoma, Burkitt's lymphoma, small cell lung cancer, non-small cell lung cancer, squamous cell lung cancer, Lewis lung carcinoma, mesothelioma, adenocarcinoma of the lung, squamous cell carcinoma, large-cell undifferentiated carcinoma, metastatic lung cancer, adenosquamous carcinoma of the lung, large cell neuroendocrine carcinoma, salivary gland-type lung carcinoma, lung carcinoids, sarcomatoid carcinoma of the lung, and malignant granular cell lung tumors.

In some embodiments, the cancer is a hematological malignancy. In some embodiments, the cancer is resistant to chemotherapy (e.g., an oxidative phosphorylation inhibitor). In some embodiments, the cancer is resistant to chemotherapy (e.g., a Bcl-2 inhibitor). In some embodiments, the resistant cancer is relapsed or refractory (r/r). In some embodiments, the cancer is a relapsed/refractory hematological malignancy. In some embodiments, the hematological malignancy is acute myeloid leukemia (AML), chronic myeloid leukemia (CIVIL), chronic myelomonocytic leukemia, thrombolytic leukemia, a myelodysplasia syndrome (MDS), a myeloproliferative disorder, refractory anemia, a preleukemia syndrome, a lymphoid leukemia, lymphoma, non-Hodgkin's lymphoma, or an undifferentiated leukemia. In some specific embodiments, the cancer is a myelodysplasia syndrome (MDS) or acute myeloid leukemia (AML). In some embodiments, the cancer is acute myeloid leukemia (AML). In some embodiments, the AML is resistant to chemotherapy (e.g., an oxidative phosphorylation inhibitor). In some embodiments, the cancer is a relapsed/refractory acute myeloid leukemia (r/r AML). In some embodiments, the cancer is a relapsed/refractory acute monocytic leukemia (r/r AMoL). In some embodiments, the cancer is a non-Hodgkin's lymphoma (NHL). In some embodiments, the cancer is a relapsed/refractory non-Hodgkin's lymphoma (r/r NHL). Non-limiting examples of non-Hodgkin's lymphoma include diffuse large B-cell lymphoma (DLBCL), mantle cell lymphoma (MCL), acute lymphocytic leukemia (ALL), and chronic lymphocytic leukemia (CLL). In some embodiments, the relapsed/refractory non-Hodgkin's lymphoma is r/r DLBCL, r/r MCL, r/r ALL, or r/r CLL.

Other exemplary cancers that may be treated by the methods described herein include but are not limited to leukemias such as erythroleukemia, acute promyelocytic leukemia, acute myeloid leukemia, acute lymphocytic leukemia, acute T-cell leukemia and lymphoma such as B-cell lymphoma (e.g. Burkitt's lymphoma), cutaneous T-cell lymphoma (CTCL), and peripheral T-cell lymphoma.

### Formulation

Some embodiments provided herein describe a pharmaceutical composition, wherein the composition further comprises one or more pharmaceutical carriers, excipients, auxiliaries, binders and/or diluents. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Any composition described herein optionally comprises minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. In some embodiments, the composition further comprises one or more of lactose, dextrose, mannitol, pH buffering agents, antioxidant agents, preservative agents, tonicity adjusters or a combination thereof. Examples of pharmaceutically acceptable carriers that are optionally used include, but are not limited to aqueous vehicles, nonaqueous vehicles, antimicrobial agents, local anesthetics, suspending and dispersing agents, emulsifying agents, sequestering or chelating agents and other pharmaceutically acceptable substances.

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate.

Further, the compounds described herein, in some embodiments, are prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds of the invention and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, N+(C1-4alkyl)4, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization. The compounds described herein can be prepared as pharmaceutically acceptable salts formed when an acidic proton present in the parent compound either is replaced by a metal ion, for example an alkali metal ion, an alkaline earth ion, or an aluminum ion; or coordinates with an organic base. Base addition salts are prepared by reacting the free acid form of the compounds described herein with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like. In addition, the salt forms of the disclosed compounds can be prepared using salts of the starting materials or intermediates.

In some embodiments, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be un-coated or coated by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, or cellulose acetate butyrate may be employed as appropriate. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with a water soluble carrier, such as polyethyleneglycol or an oil medium, for example peanut oil, liquid paraffin, or olive oil.

All formulations for oral administration should be in dosages suitable for such administration. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may also be formulated in rectal compositions such as suppositories or retention enemas, e.g., containing conventional suppository bases such as cocoa butter, polyethylene glycol, or other glycerides.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Suitable pharmaceutical carriers include inert diluents or fillers, water and various organic solvents. In some embodiments, the pharmaceutical composition contains additional ingredients such as flavorings, binders, excipients and the like. Thus for oral administration, tablets containing various excipients, such as citric acid are employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. In other embodiments, solid compositions of a similar type are employed in soft and hard filled gelatin capsules. Preferred materials, therefore, include lactose or milk sugar and high molecular weight polyethylene glycols. In certain embodiments where aqueous suspensions or elixirs are desired for oral administration, the active compound therein is combined with various sweetening or flavoring agents, coloring matters or dyes and, if desired, emulsifying agents or suspending agents, together with diluents such as water, ethanol, propylene glycol, glycerin, or combinations thereof.

In some embodiments, oily suspensions are formulated by suspending the active ingredient in a vegetable oil, for example arachis oil, olive oil, sesame oil or coconut oil, or in mineral oil such as liquid paraffin. In certain embodiments, the oily suspensions contain a thickening agent, for example beeswax, hard paraffin or cetyl alcohol. In further or additional embodiments, sweetening agents such as those set forth above, and flavoring agents are added to provide a palatable oral preparation. In other embodiments, these compositions are preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. In some embodiments, additional excipients, for example sweetening, flavoring and coloring agents, are also present. In further or additional embodiments, these compositions are preserved by the addition of an anti-oxidant such as ascorbic acid.

In some embodiments, pharmaceutical compositions are in the form of oil-in-water emulsions. In some embodiments, the oily phase is a vegetable oil, for example olive oil or arachis oil, or a mineral oil, for example liquid paraffin or mixtures of these. Suitable emulsifying agents include but are not limited to naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. In further or additional embodiments, the emulsions contain sweetening agents, flavoring agents, preservatives and antioxidants.

In some embodiments, pharmaceutical compositions described herein are in the form of a sterile injectable aqueous solution. Acceptable vehicles and solvents that are employed include but are not limited to water, Ringer's solution, phosphate buffered saline solution, U.S.P. and isotonic sodium chloride solution, ethanol, and 1,3-butanediol.

In addition, sterile, fixed oils are optionally employed as a solvent or suspending medium. For this purpose any bland fixed oil is optionally employed including synthetic mono- or diglycerides. Suitable lipophilic solvents or vehicles include fatty oils such as sesame oil, or synthetic fatty acid esters, such as ethyl oleate or triglycerides, or liposomes or other microparticulate systems may be used to target the agent to blood components or one or more organs. In some embodiments, the sterile injectable preparation is a sterile injectable oil-in-water microemulsion where the active ingredient is dissolved in the oily phase. In certain embodiments, the active ingredient is first dissolved in a mixture of soybean oil and lecithin. The oil solution then introduced into a water and glycerol mixture and processed to form a microemulsion. In further or additional embodiments, the injectable solutions or microemulsions are introduced into an individual's blood-stream by local bolus injection. Alternatively, in some embodiments, it is advantageous to administer the solution or microemulsion in such a way as to maintain a constant circulating concentration of the instant compound. In order to maintain such a constant concentration, a continuous intravenous delivery device is utilized. An example of such a device is the Deltec CADD-PLUS™ model 5400 intravenous pump.

In other embodiments, the pharmaceutical composition is in the form of a sterile injectable aqueous or oleagenous suspension for intramuscular and subcutaneous administration. In further or additional embodiments, this suspension is formulated using those suitable dispersing or wetting agents and suspending agents which have been mentioned above. In some embodiments, the sterile injectable preparation is a sterile injectable solution or suspension in a non-toxic parenterally-acceptable diluent or solvent, for example as a solution in 1,3-butanediol. In addition, sterile, fixed oils are conventionally employed as a solvent or suspending medium. For this purpose, in some embodiments, any bland fixed oil is optionally employed including synthetic mono- or diglycerides. In addition, fatty acids such as oleic acid find use in the preparation of injectables.

In certain embodiments, pharmaceutical compositions are administered in the form of suppositories for rectal administration of the drug. These compositions are prepared by mixing the active ingredient with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

In some embodiments, the compounds or compositions described herein are delivered in a vesicle, such as a liposome. In further or alternative embodiments, the compounds and pharmaceutical compositions described herein are delivered in a controlled release system, or a controlled release system can be placed in proximity of the therapeutic target. In one embodiment, a pump is used.

For topical use, creams, ointments, jellies, solutions or suspensions, etc., containing an active agent is used. As used herein, topical application includes mouth washes and gargles.

In certain embodiments, pharmaceutical compositions are administered in intranasal form via topical use of suitable intranasal vehicles and delivery devices, or via transdermal routes, using transdermal skin patches. To be administered in the form of a transdermal delivery system, the dosage administration will be continuous rather than intermittent throughout the dosage regimen.

In some embodiments, the pharmaceutical composition described herein further comprises a cyclodextrin. In some embodiments, the cyclodextrin has a concentration (w/v) ranging from about 0.001% to about 50%. In other embodiments, the cyclodextrin has a concentration (w/v) ranging from about 2% to about 48%. In other embodiments, the cyclodextrin has a concentration (w/v) ranging from about 4% to about 45%. In other embodiments, the cyclodextrin has a concentration (w/v) ranging from about 10% to about 43%. In other embodiments, the cyclodextrin has a concentration (w/v) ranging from about 15% to about 40%. In other embodiments, the cyclodextrin has a concentration (w/v) ranging from about 20% to about 38%. In other embodiments, the cyclodextrin has a concentration (w/v) ranging from about 22% to about 37%. In other embodiments, the cyclodextrin has a concentration (w/v) ranging from about 25% to about 35%. In a preferred embodiment, the cyclodextrin has a concentration (w/v) ranging from about 28% to about 32%.

Some embodiments described herein provide a composition further comprising cyclodextrin, wherein the cyclodextrin has a concentration (w/v) of about 15%, 18%, 20%, 22%, 25%, 26%, 27%, 28%, 29%, 30%, 31%, 32%, 33%, 34%, 35%, 36%, 37%, or 38% when cyclodextrin derivative is SBE7-β-CD (Captisol®). In one embodiment, the cyclodextrin has a concentration (w/v) of about 30% when cyclodextrin derivative is SBE7-β-CD (Captisol®). In another embodiment, the solubility enhancer has a concentration (w/v) of about 29.4% when the cyclodextrin derivative is SBE7-β-CD (Captisol®).

Additional cyclodextrin derivatives suitable for use in intravenous compositions described herein are known in the art and are described in, e.g., U.S. Pat. Nos. 5,134,127 and 5,376,645 each of which is incorporated by reference herein for such disclosure. In addition, examples of suitable cyclodextrin derivatives are described below.

Suitable cyclodextrins and derivatives useful in certain embodiments of the compositions, methods and kits described herein include, for example, those described in Challa et al., AAPS PharmSciTech 6(2): E329-E357 (2005), U.S. Pat. Nos. 5,134,127, 5,376,645, 5,874,418, each of which is incorporated by reference herein for such disclosure. In some embodiments, suitable cyclodextrins or cyclodextrin derivatives for use in certain embodiments of the compositions, methods and kits described herein include, but are not limited to, α-cyclodextrins, β-cyclodextrins, γ-cyclodextrins, SAE-CD derivatives (e.g., SBE-α-CD, SBE-β-CD, SBE1-β-CD, SBE4-β-CD, SBE7-β-CD (Captisol®), and SBE-γ-CD) (Cydex, Inc. Lenexa, Kans.), hydroxyethyl, hydroxypropyl (including 2- and 3-hydroxypropyl) and dihydroxypropyl ethers, their corresponding mixed ethers and further mixed ethers with methyl or ethyl groups, such as methylhydroxyethyl, ethyl-hydroxyethyl and ethyl-hydroxypropyl ethers of α-, β- and γ-cyclodextrin; and the maltosyl, glucosyl and maltotriosyl derivatives of α-, β- and γ-cyclodextrin, which may contain one or more sugar residues, e. g. glucosyl or diglucosyl, maltosyl or dimaltosyl, as well as various mixtures thereof, e. g. a mixture of maltosyl and dimaltosyl derivatives. Specific cyclodextrin derivatives for use herein include hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, diethyl-β-cyclodextrin, glucosyl-α-cyclodextrin, glucosyl-β-cyclodextrin, diglucosyl-β-cyclodextrin, tri-O-methyl-β-cyclodextrin, tri-O-ethyl-β-cyclodextrin, tri-O-butyryl-β-cyclodextrin, tri-O-valeryl-β-cyclodextrin, and di-O-hexanoyl-β-cyclodextrin, as well as methyl-β-cyclodextrin, and mixtures thereof such as maltosyl-β-cyclodextrin/dimaltosyl-β-cyclodextrin. Any suitable procedure may be utilized for preparing such cyclodextrins including, e.g., those procedures described in U.S. Pat. No. 5,024,998, which is incorporated by reference herein for such disclosure. Other cyclodextrins suitable for use in certain embodiments of the compositions, methods and kits described herein include the carboxyalkyl thioether derivatives such as ORG 26054 and ORG 25969 by ORGANON (AKZO-NOBEL), hydroxybutenyl ether derivatives by EASTMAN, sulfoalkyl-hydroxyalkyl ether derivatives, sulfoalkyl-alkyl ether derivatives, and other derivatives, for example as described in U.S. Patent Application Nos. 2002/0128468, 2004/0106575, 2004/0109888, and 2004/0063663, or U.S. Pat. Nos. 6,610,671, 6,479,467, 6,660,804, or 6,509,323, each of which is specifically incorporated by reference herein for such disclosure.

Hydroxypropyl-β-cyclodextrin can be obtained from Research Diagnostics Inc. (Flanders, N.J.). Exemplary hydroxypropyl-β-cyclodextrin products include Encapsin® (degree of substitution ˜4) and Molecusol® (degree of substitution ˜8); however, embodiments including other degrees of substitution are also available and are within the scope of the present invention.

Dimethyl cyclodextrins are available from FLUKA Chemie (Buchs, CH) or Wacker (Iowa). Other derivatized cyclodextrins suitable for use in the invention include water soluble derivatized cyclodextrins. Exemplary water-soluble derivatized cyclodextrins include carboxylated derivatives; sulfated derivatives; alkylated derivatives; hydroxyalkylated derivatives; methylated derivatives; and carboxy-β-cyclodextrins, e. g., succinyl-β-cyclodextrin (SCD). All of these materials can be made according to methods known in the art and/or are available commercially. Suitable derivatized cyclodextrins are disclosed in Modified Cyclodextrins: Scaffolds and Templates for Supramolecular Chemistry (Eds. Christopher J. Easton, Stephen F. Lincoln, Imperial College Press, London, U K, 1999) and New Trends in Cyclodextrins and Derivatives (Ed. Dominique Duchene, Editions de Sante, Paris, France, 1991).

### Dosing and Treatment Regiments

In one embodiment, the inhibitors and agents described herein, or a pharmaceutically acceptable salt thereof, are used in the preparation of medicaments for the treatment of diseases or conditions. Methods for treating any of the diseases or conditions described herein in an individual in need of such treatment, involves administration of pharmaceutical compositions described herein to said individual.

In certain embodiments, the compositions described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 1 day and 1 year, including by way of example only, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, and the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In one embodiment, the dosages appropriate for a compound of Formula (II), or a pharmaceutically acceptable salt thereof, are from about 1 milligram per kilogram body weight (“mg/kg”) to about 20 mg/kg (e.g., about 1.25, 2.5, 5, 10, 15, or 20 mg/kg). In some embodiments, the dosage is about 5 mg/kg. In some embodiments, the dosage is about 10 mg/kg. In some embodiments, the dosage is about 15 mg/kg. In some embodiments, the dosage is about 20 mg/kg.

In some embodiments, the dosage or the amount of active ingredient in the dosage form is lower or higher than the ranges indicated herein, based on a number of variables in regard to an individual treatment regime. In various embodiments, the unit dosages are altered depending on a number of variables including, but not limited to, the activity of the compound used, the disease or condition to be treated, the mode of administration, the requirements of the individual subject, the severity of the disease or condition being treated, and the judgment of the practitioner.

In some embodiments, the dosage of the Bcl-2 family inhibitor is between about 5 mg and about 600 mg. In some embodiments, the dosage of the Bcl-2 family inhibitor (e.g., venetoclax) is about 10 mg, about 20 mg, about 50 mg, about 100 mg, about 200 mg, about 300 mg, or about 400 mg. In some embodiments, the Bcl-2 family inhibitor (e.g., venetoclax) is provided in a dose of about 10 mg. In some embodiments, the Bcl-2 family inhibitor (e.g., venetoclax) is provided in a dose of about 50 mg. In some embodiments, the Bcl-2 family inhibitor (e.g., venetoclax) is provided in a dose of about 100 mg. In some embodiments, the Bcl-2 family inhibitor (e.g., venetoclax) is provided in a dose of about 200 mg. In some embodiments, the Bcl-2 family inhibitor (e.g., venetoclax) is provided in a dose of about 400 mg. In some embodiments, the Bcl-2 family inhibitor (e.g., venetoclax) is provided in a dose of about 600 mg. In some embodiments, a dose of Bcl-2 family inhibitor is administered once per day. In some embodiments, a dose is administered every day. In some embodiments, a dose of Bcl-2 family inhibitor is withheld or reduced in response to toxicity or adverse side effects. In some embodiments, a period (e.g., a day) without a dose is scheduled (e.g. a drug holiday). In some embodiments, the Bcl-2 inhibitor is administered once daily, interrupted by a drug holiday. In some embodiments, a drug holiday is one day. In some embodiments, a drug holiday is two days. In some embodiments, a drug holiday is three days. In some embodiments, a drug holiday is seven days. In some embodiments, the duration of a drug holiday is determined based on the presence of one or more side effects. In some embodiments, the Bcl-2 inhibitor is withheld until dose-limiting toxicity or side effects are reduced or eliminated. In some embodiments, doses are administered following a ramp-up schedule (e.g., doses increase over time until reaching a maximal dose, then are held consistent for a period of time thereafter). In some embodiments, a dose is increased weekly from 20 mg to 50 mg to 100 mg to 200 mg then finally 400 mg, wherein the dose is held constant at 400 mg thereafter pending toxicity or adverse effects. In any of the aforementioned aspects are further embodiments in which the effective amount of the compound described herein, or a pharmaceutically acceptable salt thereof, is: (a) systemically administered to the individual; and/or (b) administered orally to the individual; and/or (c) intravenously administered to the individual; and/or (d) administered by injection to the individual; and/or (e) administered topically to the individual; and/or (f) administered non-systemically or locally to the individual.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the individual every 8 hours; (iv) the compound is administered to the individual every 12 hours; (v) the compound is administered to the individual every 24 hours. In further or alternative embodiments, the method comprises a drug holiday, wherein the administration of the compound is temporarily suspended, or the dose of the compound being administered is temporarily reduced; at the end of the drug holiday, dosing of the compound is resumed. In one embodiment, the length of the drug holiday varies from 1 day to 1 year. In some embodiments, the combination is administered with both agents simultaneously. In some embodiments, the combination is administered with each agent separately. In some embodiments, the oxidative phosphorylation inhibitor (e.g., the “mitochondrial inhibitor,” “benzopyran derivative,” or “compound of formula (II), or pharmaceutically acceptable salt thereof”) is administered before the Bcl-2 family inhibitor. In some embodiments, a compound of formula (II), or pharmaceutically acceptable salt thereof, is administered before the Bcl-2 inhibitor. In some embodiments, a compound of formula (II), or pharmaceutically acceptable salt thereof, is administered within about 6 hours to about 10 hours of the Bcl-2 inhibitor. In some embodiments, a compound of formula (II), or pharmaceutically acceptable salt thereof, is administered within about 8 hours of the Bcl-2 inhibitor. In some embodiments, a compound of formula (II), or pharmaceutically acceptable salt thereof, is administered about 8 hours prior to the Bcl-2 inhibitor.

In some embodiments, the oxidative phosphorylation inhibitor (e.g., benzopyran derivative), or pharmaceutically acceptable salt thereof, is administered to a subject three times per week and the Bcl-2 family inhibitor is administered to the subject daily.

In some embodiments, the pharmaceutical compositions described herein are in unit dosage forms suitable for single administration of precise dosages. In unit dosage form, the formulation is divided into unit doses containing appropriate quantities of one or more active ingredient. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

Some embodiments described herein provide a pharmaceutical composition comprising a compound of Formula (II), or an enantiomer thereof, as described herein, for use in combination with a pharmaceutical composition comprising a Bcl-2 family inhibitor, as described herein, for the treatment of cancer, as described herein.

Some embodiments described herein provide a pharmaceutical composition comprising a compound of Formula (II), or an enantiomer thereof, as described herein, and a Bcl-2 family inhibitor, as described herein, for the treatment of cancer, as described herein.

Some embodiments provide a pharmaceutical composition comprising a Bcl-2 family inhibitor for use in combination with a pharmaceutical composition comprising a compound of Formula (II), or an enantiomer thereof, as described herein, for the treatment of cancer, as described herein.

Some preferred embodiments include a pharmaceutical composition comprising a compound of Formula (II) for use in combination with a pharmaceutical composition comprising a Bcl-2 family inhibitor, as described herein, for the treatment of cancer, as described herein. In some preferred embodiments, there is provided a pharmaceutical composition comprising 3-(4-hydroxyphenyl)-4-(4-hydroxyphenyl)-8-methylchroman-7-ol for use in combination with a pharmaceutical composition comprising a Bcl-2 family inhibitor, as described herein, for the treatment of cancer, as described herein. In some preferred embodiments, there is provided a pharmaceutical composition comprising cis-3-(4-hydroxyphenyl)-4-(4-hydroxyphenyl)-8-methylchroman-7-ol for use in combination with a pharmaceutical composition comprising a Bcl-2 family inhibitor, as described herein, for the treatment of cancer, as described herein. In some preferred embodiments, there is provided a pharmaceutical composition comprising d-cis-3-(4-hydroxyphenyl)-4-(4-hydroxyphenyl)-8-methylchroman-7-ol for use in combination with a pharmaceutical composition comprising a Bcl-2 family inhibitor, as described herein, for the treatment of cancer, as described herein. In some embodiments, the Bcl-2 family inhibitor is venetoclax, navitoclax, obatoclax, docetaxel, ABT-737, APG 2575, APG 1252, or AT-101, as described herein.

In certain embodiments, there is provided a pharmaceutical composition comprising a Bcl-2 family inhibitor in combination with a pharmaceutical composition comprising a d-isomer of a compound of Formula (II), as described herein, in at least, or greater than, 50%, 55%, 60%, 65%, 70%, 75%, 80%, 85%, 88%, 90%, 91%, 92%, 93%, 94%, 95%, 96%, 97%, 98%, 99%, 99.5%, or 99.9% enantiomeric excess for treatment of a disease or disorder associated with dysregulation of cell proliferation, as described herein. In certain embodiments, there is provided a pharmaceutical composition comprising Bcl-2 family inhibitor, as described herein, for combination with a pharmaceutical composition comprising 3-(4-hydroxyphenyl)-4-(4-hydroxyphenyl)-8-methylchroman-7-ol in at least, or greater than, 50%, 55%, 60%, 65%, 70%, 75%, 80%, 85%, 88%, 90%, 91%, 92%, 93%, 94%, 95%, 96%, 97%, 98%, 99%, 99.5%, or 99.9% enantiomeric excess for treatment of cancer, as described herein. In certain embodiments, there is provided a pharmaceutical composition comprising Bcl-2 family inhibitor, as described herein, for combination with a pharmaceutical composition comprising cis-3-(4-hydroxyphenyl)-4-(4-hydroxyphenyl)-8-methylchroman-7-ol in at least, or greater than, 50%, 55%, 60%, 65%, 70%, 75%, 80%, 85%, 88%, 90%, 91%, 92%, 93%, 94%, 95%, 96%, 97%, 98%, 99%, 99.5%, or 99.9% enantiomeric excess for treatment of cancer, as described herein. In certain embodiments, there is provided a pharmaceutical composition comprising Bcl-2 family inhibitor, as described herein, for combination with a pharmaceutical composition comprising d-cis-3-(4-hydroxyphenyl)-4-(4-hydroxyphenyl)-8-methylchroman-7-ol in at least, or greater than, 50%, 55%, 60%, 65%, 70%, 75%, 80%, 85%, 88%, 90%, 91%, 92%, 93%, 94%, 95%, 96%, 97%, 98%, 99%, 99.5%, or 99.9% enantiomeric excess for treatment of cancer, as described herein. In some embodiments the Bcl-2 family inhibitor is venetoclax (ABT-199, Venclexta).

Some embodiments described herein provide use of a compound of Formula (II), or an enantiomer thereof, as described herein, for the manufacture of a medicament for use in combination with a pharmaceutical composition comprising a Bcl-2 family inhibitor, as described herein, for the treatment of cancer, as described herein. In some embodiments, the Bcl-2 family inhibitor is venetoclax.

Some embodiments described herein provide use of a compound of Formula (II), or an enantiomer thereof, as described herein, and a Bcl-2 family inhibitor, for the manufacture of a medicament for use in the treatment of a cancer, as described herein.

Some embodiments described herein provide use of a Bcl-2 family inhibitor for the manufacture of a medicament for use in combination with a pharmaceutical composition comprising a compound of Formula (II), or an enantiomer thereof, as described herein, for the treatment of cancer, as described herein.

Some preferred embodiments include use of a compound of Formula (II), or an enantiomer thereof, for manufacture of a medicament for use in combination with a pharmaceutical composition comprising a Bcl-2 family inhibitor, as described herein, for the treatment of cancer, as described herein. In some embodiments, the medicament comprises 3-(4-hydroxyphenyl)-4-(4-hydroxyphenyl)-8-methylchroman-7-ol. In some embodiments, the medicament comprises cis-3-(4-hydroxyphenyl)-4-(4-hydroxyphenyl)-8-methylchroman-7-ol. In some embodiments, the medicament comprises d-cis-3-(4-hydroxyphenyl)-4-(4-hydroxyphenyl)-8-methylchroman-7-ol.

### EXAMPLES

The following examples are provided for illustrative purposes only and not to limit the scope of the claims provided herein.

### Materials and Methods

AML cell lines used as described herein include MOLM-13, MV4-11, and THP-1. MOLM-13 cells are purchased from AddexBio (San Diego, Calif., USA). MV4-11 and THP-1 cell lines are purchased from the American Type Culture Collection (Manassas, Va., USA). The cell lines are cultured in RPMI 1640 with 10-20% fetal bovine serum (Thermo Fisher Scientific, Waltham, Mass., USA), 2 mM L-glutamine, 100 U/mL penicillin, and 100 μg/mL streptomycin. All cells are cultured in a 37° C. humidified atmosphere containing 5% CO₂/95% air. The cell lines are authenticated at the Genomics Core at Karmanos Cancer Institute using the Power-Plex 16 System from Promega (Madison, Wis., USA). Cell lines are tested for the presence of Mycoplasma by PCR on a monthly basis.

### Example 1: Venetoclax+Compound A Induces Cell Death, Decreases Viability in AML Cell Lines

Human adult AML cell lines (MOLM-13), human childhood AML cell lines (MV4-11 and THP-1), and cytarabine-resistant (araC-R) acute monocytic leukemia (AMoL) cell lines (U937) are treated with venetoclax and d-cis-3-(4-hydroxyphenyl)-4-(4-hydroxyphenyl)-8-methylchroman-7-ol (Compound A), alone or in combination, and are subject to flow cytometry analysis using Annexin V-fluorescein isothiocyanate (FITC)/propidium iodide (PI) Apoptosis Kit (Beckman Coulter, Brea, Calif.). Results are expressed as percent Annexin V-positive (Annexin V+) cells. For the AML/AMoL cell lines, experiments are performed three independent times in triplicate, and the data presented are from one representative experiment. The extent and direction of the antileukemic interaction between venetoclax and Compound A is determined by calculating the combination index (CI). CI<1 indicates synergistic effects, CI=1 indicates additive effects, and CI>1 indicates antagonistic effects. ***indicates p<0.001 compared to control.

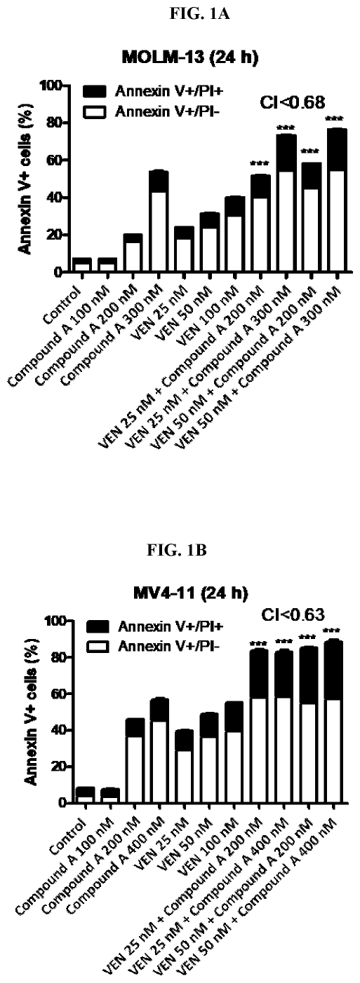

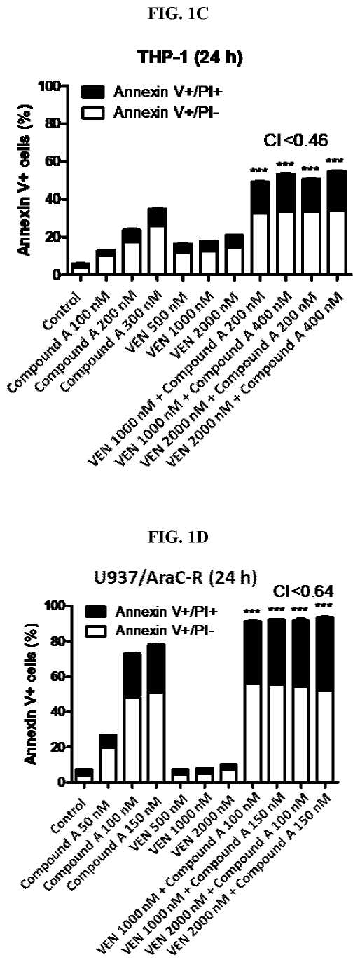

Antileukemic activity of venetoclax and Compound A, alone or in combination, is evaluated in MOLM-13, MV4-11, THP-1, and U937 cell lines at clinically achievable concentrations. Single drug treatment induces high levels of apoptosis, measured by Annexin V+ staining and flow cytometry analyses. And yet, when venetoclax and Compound A are combined, apoptosis is significantly increased (p<0.001) relative to any single drug treatment. Additionally, necrosis as measured by staining with propidium iodide (PI+), is included as an additional measure of cell death in cancer cell lines. Synergism is observed with the combination of venetoclax and Compound A in every cell line tested. CI in the MOLM-13 AML cell line is <0.68 (FIG. 1A), CI in the MV4-11 AML cell line is <0.63 (FIG. 1B), CI in the THP-1 AML cell line is <0.46 (FIG. 1C), and CI in the cytarabine-resistant U937 cell line is <0.64 (FIG. 1D).

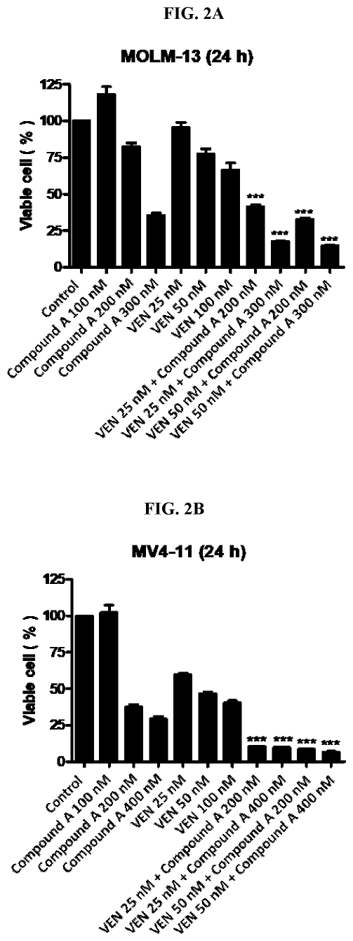

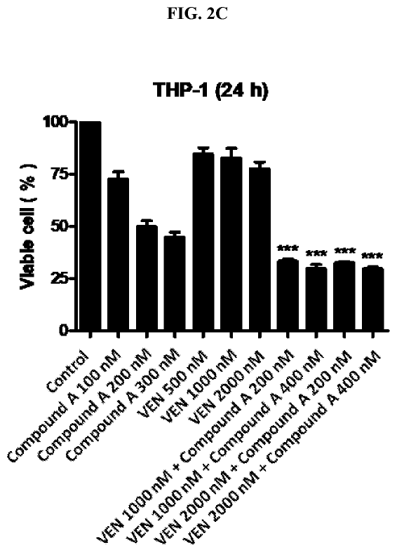

Viability is also determined in MOLM-13 AML cells (FIG. 2A), MV4-11 AML cells (FIG. 2B), and THP-1 AML cells (FIG. 2C) for venetoclax and Compound A, alone or combined, at clinically achievable concentrations. While single drug treatment generally shows a dose-dependent decrease in viability compared to control, the combination of venetoclax+Compound A provided a statistically significant reduction (p<0.001) in viability in all three cell lines evaluated after 24 hr in every combination tested.

### Example 2: Venetoclax+Compound A in H596 Squamous Lung Cancer Cell Lines

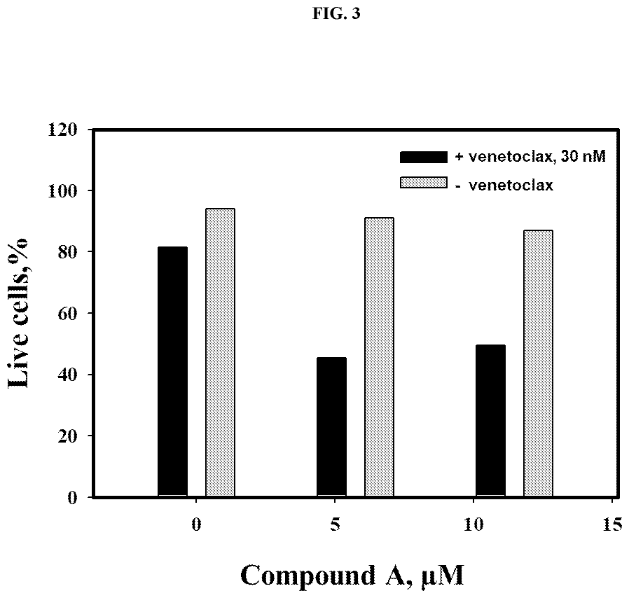

H596 squamous lung cancer cells are treated with Compound A at concentrations of 0 μM (control), 5 μM and 10 μM, either alone or with 30 nM venetoclax. After 48 h, viability, as measured by percent live cells, is evaluated at each concentration of Compound A, both with and without venetoclax. Viability was determined by MTT [3-(4,5-dimethylthiazol-2-yl)-2,5-diphenyltetrazolium bromide] assay. Briefly, MTT solution in 1×PBS is added to each well at the final concentration of 0.5 mg/mL. The plate is incubated for 4 h at 37° C. The MTT medium is aspirated carefully and the dark-blue formazan is solubilized in DMSO (Sigma-Aldrich). Optical density is measured with a spectrometer (BioRad, Hercules, Calif.) at 550/690 nm. Each experiment is conducted in triplicates and repeated independently 3 times. Results (% viability) are calculated relative to untreated control cells. Synergistic reduction in cell viability is observed for the combination of 30 nM venetoclax+5 μM and 10 μM concentrations of Compound A relative to control. Whereas the 30 nM dose of venetoclax was not effective at reducing viability in the control (0 μM Compound A), this combination was effective in 5 μM and 10 μM concentrations of Compound A. H596 cells have been previously found to be resistant to monotherapy treatment with a compound such as Compound A. These data suggest a synergistic benefit for the combination of venetoclax and Compound A, even in drug-resistant cell lines, as this combination was superior to venetoclax alone (FIG. 3).

What is claimed is:

1. A method of treating cancer, comprising administering to a subject in need thereof an effective amount of:
(i) a Bcl-2 family inhibitor; and
(ii) a compound of formula (II) or a pharmaceutically acceptable salt thereof: 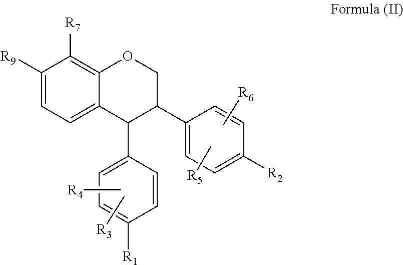
wherein
R₁ is hydroxy, alkoxy, haloalkyl, or halo;
R₂ is hydroxy or alkoxy;
R₃, R₄, R₅, and R₆ are independently hydrogen, hydroxy, alkoxy, halo, haloalkyl, or alkyl;
R₇ is alkyl or hydrogen; and
R₉ is hydroxy or alkoxy.

2. The method of claim 1, wherein R₁ is hydroxy or alkoxy.

3. The method of either of claim 1 or 2, wherein R₂ is hydroxy.

4. The method of any one of claims 1-3, wherein R₃, R₄, R₅, and R₆ are independently hydrogen or alkyl.

5. The method of any one of claims 1-4, wherein R₃, R₄, R₅, and R₆ are independently hydrogen.

6. The method of any one of claims 1-5, wherein R₇ is methyl or hydrogen.

7. The method of claim 1, wherein
R₁ is hydroxy or alkoxy;
R₂ is hydroxy or alkoxy;
R₃, R₄, R₅, and R₆ are independently hydrogen, hydroxy, alkoxy, or alkyl;
R₇ is alkyl or hydrogen; and
R₉ is hydroxy or alkoxy.

8. The method of claim 1, wherein
R₁ is hydroxy or alkoxy;
R₂ is hydroxy or alkoxy;
R₃, R₄, R₅, and R₆ are independently hydrogen;
R₇ is alkyl or hydrogen; and
R₉ is hydroxy.

9. The method of claim 1, wherein
R₁ is hydroxy or methoxy;
R₂ is hydroxy or methoxy;
R₃, R₄, R₅, and R₆ are independently hydrogen, hydroxy, methoxy, methyl;
R₇ is methyl or hydrogen; and
R₉ is hydroxy or methoxy.

10. The method of claim 1, wherein
R₁ is hydroxy or methoxy;
R₂ is hydroxy or methoxy;
R₃, R₄, R₅, and R₆ are independently hydrogen;
R₇ is methyl or hydrogen; and
R₉ is hydroxy.

11. The method of claim 1, wherein the compound of formula (II) is 3-(4-hydroxyphenyl)-4-(4-hydroxyphenyl)-8-methylchroman-7-ol.

12. The method of claim 1, wherein the compound of formula (II) is cis-3-(4-hydroxyphenyl)-4-(4-hydroxyphenyl)-8-methylchroman-7-ol.

13. The method of claim 1, wherein the compound of formula (II) is d-cis-3-(4-hydroxyphenyl)-4-(4-hydroxyphenyl)-8-methylchroman-7-ol.

14. The method of any one of claims 1-13, wherein the Bcl-2 family inhibitor is an inhibitor of Bcl-2, Bcl-xL, Bcl-x, Bcl-w, Bcl-b, BH3-only, or MCL-1.

15. The method of any one of claims 1-14, wherein the Bcl-2 family inhibitor is an inhibitor of Bcl-2.

16. The method of any one of claims 1-15, wherein the Bcl-2 family inhibitor is a BH3-mimetic.

17. The method of any one of claims 1-16, wherein the Bcl-2 family inhibitor is venetoclax, navitoclax, obatoclax mesylate, ABT-737, APG 2575, APG 1252, or AT-101.

18. The method of any one of claims 1-17, wherein the Bcl-2 family inhibitor is venetoclax.

19. The method of any one of claims 1-18, wherein the cancer is leukemia, lymphoma, lung cancer, or a hematological malignancy.

20. The method of any one of claims 1-19, wherein the cancer is a leukemia or lymphoma.

21. The method of any one of claims 1-20, wherein the leukemia is acute myeloid leukemia (AML), chronic myeloid leukemia (CIVIL), acute lymphocytic leukemia (ALL), or chronic lymphocytic leukemia (CLL).

22. The method of claim 21, wherein the leukemia is acute myeloid leukemia (AML).

23. The method of any one of claims 1-19, wherein the cancer is lung cancer.

24. The method of claim 23, wherein the lung cancer is non-small cell lung cancer (NSCLC), small cell lung cancer (SCLC), a lung carcinoid tumor, or adenoid cystic carcinoma.

25. The method of claim 24, wherein the non-small cell lung cancer (NSCLC) is lung adenocarcinoma, squamous cell carcinoma, large cell (undifferentiated) carcinoma, adenosquamous carcinoma, or sarcomatoid carcinoma.

26. The method of any one of claims 1-25, wherein the cancer is non-responsive or resistant to the Bcl-2 inhibitor.

27. The method of any one of claims 1-26, wherein the Bcl-2 family inhibitor and compound of formula (II) are administered simultaneously.

28. The method of any one of claims 1-26, wherein the Bcl-2 family inhibitor and compound of formula (II) are administered sequentially.

29. A method of treating leukemia comprising administering to a subject in need thereof an effective amount of:
(i) a Bcl-2 family inhibitor; and
(ii) a compound of formula (II) or a pharmaceutically acceptable salt thereof: 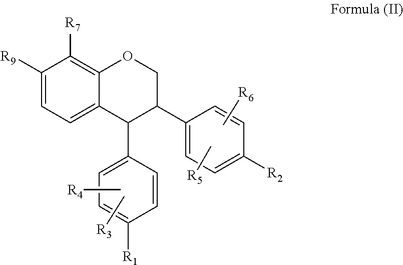
wherein
R₁ is hydroxy, alkoxy, haloalkyl, or halo;
R₂ is hydroxy or alkoxy;
R₃, R₄, R₅, and R₆ are independently hydrogen, hydroxy, alkoxy, halo, haloalkyl, or alkyl;
R₇ is alkyl or hydrogen; and
R₉ is hydroxy or alkoxy.

30. The method of claim 29, wherein the leukemia is acute myeloid leukemia (AML), chronic myeloid leukemia (CML), acute lymphocytic leukemia (ALL), or chronic lymphocytic leukemia (CLL).

31. The method of claim 29 or 30, wherein the leukemia is acute myeloid leukemia (AML).

32. The method of any one of claims 29-31, wherein the Bcl-2 family inhibitor is venetoclax, navitoclax, obatoclax mesylate, ABT-737, APG 2575, APG 1252, or AT-101.

33. The method of any one of claims 29-32, wherein the Bcl-2 family inhibitor is venetoclax.

34. The method of any one of claims 29-33, wherein the compound of formula (II) is 3-(4-hydroxyphenyl)-4-(4-hydroxyphenyl)-8-methylchroman-7-ol.

35. The method of any one of claims 29-33, wherein the compound of formula (II) is cis-3-(4-hydroxyphenyl)-4-(4-hydroxyphenyl)-8-methylchroman-7-ol.

36. The method of any one of claims 29-33, wherein the compound of formula (II) is d-cis-3-(4-hydroxyphenyl)-4-(4-hydroxyphenyl)-8-methylchroman-7-ol.

37. A method of treating lung cancer comprising administering to a subject in need thereof an effective amount of:
(i) a Bcl-2 family inhibitor; and
(ii) a compound of formula (II) or a pharmaceutically acceptable salt thereof: 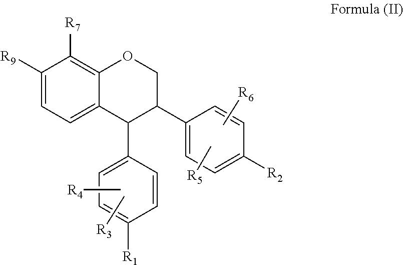
wherein
R₁ is hydroxy, alkoxy, haloalkyl, or halo;
R₂ is hydroxy or alkoxy;
R₃, R₄, R₅, and R₆ are independently hydrogen, hydroxy, alkoxy, halo, haloalkyl, or alkyl;
R₇ is alkyl or hydrogen; and
R₉ is hydroxy or alkoxy.

38. The method of claim 7, wherein the lung cancer is non-small cell lung cancer (NSCLC), small cell lung cancer (SCLC), a lung carcinoid tumor, or adenoid cystic carcinoma.

39. The method of claim 38, wherein the non-small cell lung cancer (NSCLC) is lung adenocarcinoma, squamous cell carcinoma, large cell (undifferentiated) carcinoma, adenosquamous carcinoma, or sarcomatoid carcinoma.

40. The method of any one of claims 37-39, wherein the Bcl-2 family inhibitor is venetoclax, navitoclax, obatoclax mesylate, ABT-737, APG 2575, APG 1252, or AT-101.

41. The method of any one of claims 37-40, wherein the Bcl-2 family inhibitor is venetoclax.

42. The method of any one of claims 37-41, wherein the compound of formula (II) is 3-(4-hydroxyphenyl)-4-(4-hydroxyphenyl)-8-methylchroman-7-ol.

43. The method of any one of claims 37-41, wherein the compound of formula (II) is cis-3-(4-hydroxyphenyl)-4-(4-hydroxyphenyl)-8-methylchroman-7-ol.

44. The method of any one of claims 37-41, wherein the compound of formula (II) is d-cis-3-(4-hydroxyphenyl)-4-(4-hydroxyphenyl)-8-methylchroman-7-ol.

45. A pharmaceutical composition comprising:
(i) a Bcl-2 family inhibitor; and
(ii) a compound of formula (II) or a pharmaceutically acceptable salt thereof: 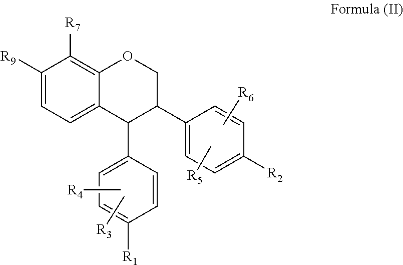
wherein
R₁ is hydroxy, alkoxy, haloalkyl, or halo;
R₂ is hydroxy or alkoxy;
R₃, R₄, R₅, and R₆ are independently hydrogen, hydroxy, alkoxy, halo, haloalkyl, or alkyl;
R₇ is alkyl or hydrogen; and
R₉ is hydroxy or alkoxy; and
(iii) a pharmaceutically acceptable excipient.

46. The pharmaceutical composition of claim 45, wherein the Bcl-2 family inhibitor is venetoclax, navitoclax, obatoclax mesylate, ABT-737, APG 2575, APG 1252, or AT-101.

47. The pharmaceutical composition of claim 45 or 46, wherein the Bcl-2 family inhibitor is venetoclax.

48. The pharmaceutical composition of any one of claims 45-47, wherein the compound of formula (II) is 3-(4-hydroxyphenyl)-4-(4-hydroxyphenyl)-8-methylchroman-7-ol.

49. The pharmaceutical composition of any one of claims 45-47, wherein the compound of formula (II) is cis-3-(4-hydroxyphenyl)-4-(4-hydroxyphenyl)-8-methylchroman-7-ol.

50. The pharmaceutical composition of any one of claims 45-47, wherein the compound of formula (II) is d-cis-3-(4-hydroxyphenyl)-4-(4-hydroxyphenyl)-8-methylchroman-7-ol.

51. A kit comprising one or more containers filled with a Bcl-2 family inhibitor and one or more containers filled with a compound of formula (II) or a pharmaceutically acceptable salt thereof: 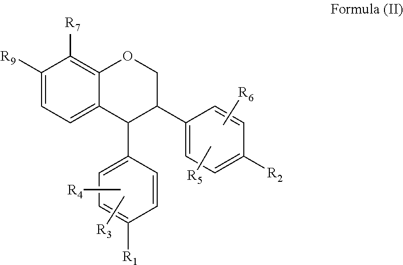
wherein
R₁ is hydroxy, alkoxy, haloalkyl, or halo;
R₂ is hydroxy or alkoxy;
R₃, R₄, R₅, and R₆ are independently hydrogen, hydroxy, alkoxy, halo, haloalkyl, or alkyl;
R₇ is alkyl or hydrogen; and
R₉ is hydroxy or alkoxy.

52. The kit of claim 51, wherein the Bcl-2 inhibitor is venetoclax.

53. The kit of claim 51 or 52, wherein the at least one compound of formula (II) is 3-(4-hydroxyphenyl)-4-(4-hydroxyphenyl)-8-methylchroman-7-ol.

54. The kit of claim 51 or 52, wherein the at least one compound of formula (II) is cis-3-(4-hydroxyphenyl)-4-(4-hydroxyphenyl)-8-methylchroman-7-ol.

55. The kit of claim 51 or 52, wherein the at least one compound of formula (II) is d-cis-3-(4-hydroxyphenyl)-4-(4-hydroxyphenyl)-8-methylchroman-7-ol.

56. A compound of formula (II) or a pharmaceutically acceptable salt thereof: 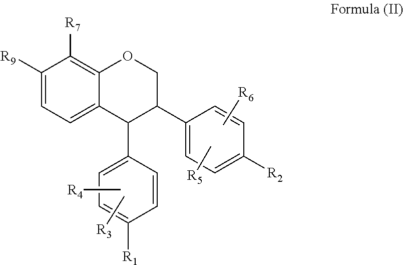
wherein
R₁ is hydroxy, alkoxy, haloalkyl, or halo;
R₂ is hydroxy or alkoxy;
R₃, R₄, R₅, and R₆ are independently hydrogen, hydroxy, alkoxy, halo, haloalkyl, or alkyl;
R₇ is alkyl or hydrogen; and
R₉ is hydroxy or alkoxy;
for use in combination with a Bcl-2 family inhibitor.

57. The compound of claim 56, for use in the treatment of cancer.

58. The compound of claim 56, for use in the treatment of leukemia, lymphoma, lung cancer, or a hematological malignancy.

59. The compound of claim 56, for use in the treatment of a leukemia or lymphoma.

60. The compound of claim 56, for use in the treatment of acute myeloid leukemia (AML), chronic myeloid leukemia (CIVIL), acute lymphocytic leukemia (ALL), or chronic lymphocytic leukemia (CLL).

61. The compound of claim 56, for use in the treatment of acute myeloid leukemia (AML).

62. The compound of claim 56, for use in the treatment of acute myeloid leukemia (AML) that is relapsed or refractory (r/r AML).

63. The compound of claim 56, for use in the treatment of AML or r/r AML, wherein the AML or r/r AML is resistant to standard of care therapy.

64. The compound of claim 62, wherein the AML or r/r AML is resistant to cytarabine, venetoclax, or an oxidative phosphorylation inhibitor.

65. The compound of claim 56, wherein the compound of formula (II) is 3-(4-hydroxyphenyl)-4-(4-hydroxyphenyl)-8-methylchroman-7-ol.

66. The compound of claim 56, wherein the compound of formula (II) is cis-3-(4-hydroxyphenyl)-4-(4-hydroxyphenyl)-8-methylchroman-7-ol.

67. The compound of claim 56, wherein the compound of formula (II) is d-cis-3-(4-hydroxyphenyl)-4-(4-hydroxyphenyl)-8-methylchroman-7-ol.

68. The compound of any one of claims 56-67, wherein the Bcl-2 family inhibitor is venetoclax.
